(19) United States
(12) Patent Application Publication
(10) Pub. No.: US 2006/0293266 A1
(43) Pub. Date: Dec. 28, 2006

(54) Phosphodiesterase 4D in the ryanodine receptor complex protects against heart failure

(75) Inventor: Andrew R. Marks, Larchmont, NY (US)

Correspondence Address: THELEN REID & PRIEST LLP;LESLIE G. RESTAINO, 200 CAMPUS DRIVE, SUITE 210, FLORHAM PARK, NJ, 07932, US

(73) Assignee: THE TRUSTEES OF COLUMBIA, New York, NY (US)

(21) Appl. No.: 11/305,528

(22) Filed: Dec. 16, 2005

Related U.S. Application Data
(63) Continuation-in-part of application No. 10/794,218, filed on Mar. 5, 2004.
(63) Continuation-in-part of application No. 10/608,723, filed on Jun. 26, 2003.
Application No. 10/608,723 is a continuation-in-part of application No. 10/288,606, filed on Nov. 5, 2002.
Application No. 10/288,606 is a continuation of application No. 09/568,474, filed on May 10, 2000, now Pat. No. 6,489,125.
(60) Provisional application No. 60/636,959, filed on Dec. 16, 2004.
(60) Provisional application No. 60/452,664, filed on Mar. 7, 2003.

(51) Int. Cl. A61K 48/00; A61K 31/519

(52) U.S. Cl. 514/44; 514/252.16; 514/262.1

(57) ABSTRACT

The present invention provides compositions useful for treating and preventing ryanodine receptor associated disorders comprising a PDE-associated agent and a pharmaceutically acceptable carrier. The present invention also provides methods for treating or preventing ryanodine receptor associated disorders including cardiac disorders and diseases, skeletal muscular disorders and diseases, cognitive disorders and diseases malignant hyperthermia, diabetes and sudden infant death syndrome. The present invention further provides methods for regulating PKA phosphorylation of a ryanodine receptor as well as methods for regulating Ca+2 release and reuptake in cells. Also provided are kits for use in delivering a PDE-associated agent to cardiac cells in a subject, comprising the composition of the present invention and a catheter.

### CROSS REFERENCE TO RELATED APPLICATIONS

This application claims the benefit of U.S. Provisional Application Ser. No. 60/636,959, filed Dec. 16, 2004, and is a continuation-in-part of U.S. application Ser. No. 10/794,218, filed Mar. 5, 2004; which claims the benefit of U.S. Provisional Application Ser. No. 60/452,664, filed Mar. 7, 2003. This application is also a continuation-in-part application of U.S. application Ser. No. 10/608,723, filed Jun. 26, 2003, which is a continuation-in-part of U.S. application Ser. No. 10/288,606, filed on Nov. 5, 2002; which is a continuation of U.S. patent application Ser. No. 09/568,474, filed on May 10, 2000, now U.S. Pat. No. 6,489,125, which issued on Dec. 3, 2002. The contents of each of these applications are incorporated herein in their entirety by reference thereto.

### STATEMENT OF GOVERNMENT INTEREST

This invention was made with government support under NIH Grant No. NIH-RO1-HD20788. As such, the United States government may have certain rights in this invention.

### FIELD OF THE INVENTION

This invention relates to novel compositions and methods to treat and prevent disorders and diseases associated with the RyR receptors that regulate calcium channel functioning in cells.

### BACKGROUND OF THE INVENTION

Througout this application, various publications are referenced in parentheses by author and year. Full citations for these references are provided at the end of the specification immediately preceding the claims. The disclosures of these publications in their entireties are hereby incorporated by reference into this application to more fully describe the state of the art to which this invention pertains.

The sarcoplasmic reticulum (SR) is a structure in cells that functions, among other things, as a specialized intracellular calcium (Ca²⁺) store. Channels in the SR called ryanodine receptors (RyRs) open and close to regulate the release of Ca²⁺ from the SR into the intracellular cytoplasm of the cell. Release of Ca²⁺ into the cytoplasm from the SR increases cytoplasmic Ca²⁺ concentration. Open probability (Po) of the RyR receptor refers to the likelihood that the RyR channel is open at any given moment, and therefore capable of releasing Ca²⁺ into the cytoplasm from the SR.

There are three types of ryanodine receptors, all of which are highly-related Ca²⁺ channels: RyR1, RyR2, and RyR3. RyR1 is found predominantly in skeletal muscle as well as other tissues, RyR2 is found predominantly in the heart as well as other tissues, and RyR3 is found in the brain as well as other tissues. The RyR channels are formed by four RyR polypeptides in association with four FK506 binding proteins (FKBPs), specifically FKBP12 (calstabin1) and FKBP12.6 (calstabin2). Calstabin1 binds to RyR1, calstabin2 binds to RyR2, and calstabin1 binds to RyR3. The FKBP proteins (calstabin1 and calstabin2) bind to the RyR channel (one molecule per RyR subunit), stabilize RyR-channel functioning, and facilitate coupled gating between neighboring RyR channels, thereby preventing abnormal activation of the channel during the channel's closed state.

Besides the calstabin binding proteins, protein kinase A (PKA) also binds to the cytoplasmic surface of the RyR receptors. PKA phosphorylation of the RyR receptors causes partial dissociation of calstabins from RyRs. Dissociation of calstabin from RyR causes increased open probability of RyR, and therefore increased Ca²⁺ release from the SR into the intracellular cytoplasm.

Ca²⁺ release from the SR in skeletal muscle cells and heart cells is a key physiological mechanism that controls muscle performance, because increased concentration of Ca²⁺ in the intracellular cytoplasm causes contraction of the muscle.

Excitation-contraction (EC) coupling in skeletal muscles involves electrical depolarization of the plasma membrane in the transverse tubule (T-tubule), which activates voltage-gated L-type Ca²⁺ channels (LTCCs). LTCCs trigger Ca²⁺ release from the SR through physical interaction with RyR1. The resulting increase in cytoplasmic Ca²⁺ concentration induces actin-myosin interaction and muscle contraction. To enable relaxation, intracellular Ca²⁺ is pumped back into the SR via SR Ca²⁺-ATPase pumps (SERCAs), which is regulated by phospholamban (PLB) depending on the muscle fiber type.

It has been shown that disease forms that result in sustained activation of the sympathetic nervous system and increased plasma catecholamine levels cause maladaptive activation of intracellular stress pathways resulting in destabilization of the RyR1 channel closed state and intracellular Ca²⁺ leak. SR Ca²⁺ leak via RyR1 channels was found to deplete intracellular SR calcium stores, to increase compensatory energy consumption, and to result in significant acceleration of muscle fatigue. The stress-induced muscle defect permanently reduces isolated muscle and in vivo performance particularly in situations of increased demand.

It also has been shown that destabilization of the RyR1 closed state occurs under pathologic conditions of increased sympathetic activation and involves depletion of the stabilizing calstabin1 (FKBP12) channel subunit. Proof-of-principle experiments have shown that PKA activation as an end effector of the sympathetic nervous systems increases RyR1 PKA phosphorylation at Ser-2843 which decreases the binding affinity of calstabin1 to RyR1 and increases channel open probability.

In cardiac striated muscle, RyR2 is the major Ca²⁺-release channel required for EC coupling and muscle contraction. During EC coupling, depolarization of the cardiac-muscle cell membrane during phase zero of the action potential activates voltage-gated Ca²⁺ channels. Ca²⁺ influx through the open voltage-gated channels in turn initiates Ca²⁺ release from the SR via RyR2. This process is known as Ca²⁺-induced Ca²⁺ release. The RyR2-mediated, Ca²⁺-induced Ca²⁺ release then activates the contractile proteins in the cardiac cell, resulting in cardiac muscle contraction.

Phosphorylation of cardiac RyR2 by PKA is an important part of the “fight or flight” response that increases cardiac EC coupling gain by augmenting the amount of Ca²⁺ released for a given trigger. This signaling pathway provides a mechanism by which activation of the sympathetic nervous system, in response to stress, results in increased cardiac output. PKA phosphorylation of RyR2 increases the open probability of the channel by dissociating calstabin2 (FKBP12.6) from the channel complex. This, in turn, increases the sensitivity of RyR2 to Ca²⁺-dependent activation.

Despite advances in treatment, heart failure remains an important cause of mortality in Western countries. An important hallmark of heart failure is reduced myocardial contractility. In heart failure, contractile abnormalities result, in part, from alterations in the signaling pathway that allows the cardiac action potential to trigger Ca²⁺ release via RyR2 channels and muscle contraction. In particular, in failing hearts, the amplitude of the whole-cell Ca²⁺ transient is decreased and the duration prolonged.

Cardiac arrhythmia, a common feature of heart failure, results in many of the deaths associated with the disease. Atrial fibrillation (AF) is the most common cardiac arrhythmia in humans, and represents a major cause of morbidity and mortality. Structural and electrical remodeling—including shortening of atrial refractoriness, loss of rate-related adaptation of refractoriness, and shortening of the wavelength of re-entrant wavelets—accompany sustained tachycardia. This remodeling is likely important in the development, maintenance and progression of atrial fibrillation. Studies suggest that calcium handling plays a role in electrical remodeling in atrial fibrillation.

Approximately 50% of all patients with heart disease die from fatal cardiac arrhythmias. In some cases, a ventricular arrhythmia in the heart is rapidly fatal—a phenomenon referred to as “sudden cardiac death” (SCD). Fatal ventricular arrhythmias and SCD also occur in young, otherwise-healthy individuals who are not known to have structural heart disease. In fact, ventricular arrhythmia is the most common cause of sudden death in otherwise-healthy individuals.

Catecholaminergic polymorphic ventricular tachycardia (CPVT) is an inherited disorder in individuals with structurally normal hearts. It is characterized by stress-induced ventricular tachycardia—a lethal arrhythmia that causes SCD. In subjects with CPVT, physical exertion and/or stress induce bidirectional and/or polymorphic ventricular tachycardias that lead to SCD even in the absence of detectable structural heart disease. CPVT is predominantly inherited in an autosomal-dominant fashion. Individuals with CPVT have ventricular arrhythmias when subjected to exercise, but do not develop arrhythmias at rest. Studies have identified mutations in the human RyR2 gene, on chromosome 1q42-q43, in individuals with CPVT.

Failing hearts (e.g., in patients with heart failure and in animal models of heart failure) are characterized by a maladaptive response that includes chronic hyperadrenergic stimulation. In heart failure, chronic beta-adrenergic stimulation is associated with the activation of beta-adrenergic receptors in the heart, which, through coupling with G-proteins, activate adenylyl cyclase and thereby increase intracellular cAMP concentration. cAMP activates cAMP-dependent PKA, which has been shown to induce hyperphosphorylation of RyR2. Thus, chronic heart failure is a chronic hyperadrenergic state which results in several pathologic consequences, including PKA hyperphosphorylation of RyR2.

The PKA hyperphosphorylation of RyR2 has been proposed as a factor contributing to depressed contractile function and arrhythmogenesis in heart failure. Consistent with this hypothesis, PKA hyperphosphorylation of RyR2 in failing hearts has been demonstrated, in vivo, both in animal models and in patients with heart failure undergoing cardiac transplantation.

In failing hearts, the hyperphosphorylation of RyR2 by PKA induces the dissociation of FKBP12.6 (calstabin2) from the RyR2 channel. This causes marked changes in the biophysical properties of the RyR2 channel, including increased open probability (Po) due to an increased sensitivity to Ca²⁺-dependent activation; destabilization of the channel, resulting in subconductance states; and impaired coupled gating of the channels, resulting in defective EC coupling and cardiac dysfunction. Thus, PKA-hyperphosphorylated RyR2 is very sensitive to low-level Ca²⁺ stimulation, and this manifests itself as a diastolic SR Ca²⁺ leak through the PKA hyperphosphorylated RyR2 channel.

The maladaptive response to stress in heart failure results in depletion of FKBP12.6 from the channel macromolecular complex. This leads to a shift to the left in the sensitivity of RyR2 to Ca²⁺-induced Ca²⁺ release, resulting in channels that are more active at low-to-moderate Ca²⁺ concentrations. Over time, the increased “leak” through RyR2 results in resetting of the SR Ca²⁺ content to a lower level, which in turn reduces EC coupling gain and contributes to impaired systolic contractility.

Additionally, a subpopulation of RyR2 that are particularly “leaky” can release SR Ca²⁺ during the resting phase of the cardiac cycle, diastole. This results in depolarizations of the cardiomyocyte membrane known as delayed after-depolarizations (DADs), which are known to trigger fatal ventricular cardiac arrhythmias.

In patients with CPVT mutations in their RyR2 and otherwise structurally-normal hearts, a similar phenomenon is at work. Specifically, it is known that exercise and stress induce the release of catecholamines that activate beta-adrenergic receptors in the heart. Activation of the beta-adrenergic receptors leads to PKA hyperphosphorylation of RyR2 channels. Evidence also suggests that the PKA hyperphosphorylation of RyR2 resulting from beta-adrenergic-receptor activation renders mutated RyR2 channels more likely to open in the relaxation phase of the cardiac cycle, increasing the likelihood of arrhythmias.

Cardiac arrhythmias are known to be associated with diastolic SR Ca²⁺ leaks in patients with CPVT mutations in their RyR2 and otherwise structurally-normal hearts. In these cases, the most common mechanism for induction and maintenance of ventricular tachycardia is abnormal automaticity. One form of abnormal automaticity, known as triggered arrhythmia, is associated with aberrant release of SR Ca²⁺, which initiates DADs. DADs are abnormal depolarizations in cardiomyocytes that occur after repolarization of a cardiac action potential. The molecular basis for the abnormal SR Ca²⁺ release that results in DADs has not been fully elucidated. However, DADs are known to be blocked by ryanodine, providing evidence that RyR2 plays a key role in the pathogenesis of this aberrant Ca²⁺ release.

Co-pending application U.S. Ser. No. 10/763,498 discusses RyR2 as a target for treating and preventing heart failure and cardiac arrhythmias, including atrial fibrillation and cardiac arrhythmias that cause exercise-induced sudden cardiac death (SCD). RyR2 channels with 7 different CPVT mutations (e.g., S2246L, R2474S, N4104K, R4497C, P2328S, Q4201R, V4653F) were found to have functional defects that resulted in channels that became leaky (i.e., a calcium leak) when stimulated during exercise. The mechanism for the VT in CPVT has been demonstrated to be the same as the mechanism for VT in heart failure.

It has been shown that exercise-induced arrhythmias and sudden death (in patients with CPVT) result from a reduced affinity of FKBP12.6 (calstabin2) for RyR2. Additionally, it has been demonstrated that exercise activates RyR2 as a result of phosphorylation by adenosine 3′, 5′-monophosphate (cAMP)-dependent protein kinase (PKA). Mutant RyR2 channels, which had normal function in planar lipid bilayers under basal conditions, were more sensitive to activation by PKA phosphorylation—exhibiting increased activity (open probability) and prolonged open states, as compared with wild-type channels. In addition, PKA-phosphorylated mutant RyR2 channels were resistant to inhibition by Mg²⁺, a physiological inhibitor of the channel, and showed reduced binding to FKBP12.6 (aka calstabin2, which stabilizes the channel in the closed state). These findings indicate that, during exercise, when the RyR2 are PKA-phosphorylated, the mutant CPVT channels are more likely to open in the relaxation phase of the cardiac cycle (diastole), increasing the likelihood of arrhythmias triggered by SR Ca²⁺ leak.

Additionally, co-pending U.S. patent application Ser. No. 09/288,606 discusses a method for regulating contraction of a subject's heart by administering a compound which regulates PKA phosphorylation of an RyR2 receptor and specifically decreases PKA phosphorylation. Co-pending U.S. patent application Ser. No. 10/608,723 also discusses a method for treating and prophylaxis for atrial tachyarrhythmia and exercise and stress-induced arrhythmias by administration of an agent which inhibits PKA phosphorylation of RyR2.

Phosphodiesterases (PDEs) control the temporal and spatial dynamics of the second messenger 3′,5′ cyclic adenosine monophosphate (cAMP), allowing for highly localized cAMP gradients in cells (Zaccolo and Pozzan, 2002). Localization of PDEs in close proximity to cAMP-dependent protein kinase A (PKA) is thought to control access of cAMP to the regulatory kinase subunit (Conti et al., 2003 and Houslay and Adams, 2003). PKA phosphorylation of proteins mediates a wide variety of signals, including those generated during activation of the sympathetic nervous system (SNS) as part of the “fight or flight” response. On the other hand, chronic activation of the SNS is a characteristic finding in heart failure, and acute stimulation of the SNS has been linked to triggered arrhythmias associated with sudden cardiac death.

In the heart, phosphodiesterase 4 (PDE4) contributes to the regulation of cAMP levels in cardiac myocytes. In particular, PDE4 cAMP-hydrolyzing activity has been localized to the transverse (T) tubule/sarcoplasmic reticulum (SR) junctional space that is involved in excitation-contraction coupling (Mongillo et al., 2004 and Zaccolo and Pozzan, 2002). PDEs have been shown to be components of macromolecular signaling complexes via binding to targeting proteins including muscle A-kinase anchoring proteins (AKAPs) (Dodge et al., 2001). PDEs in cardiac muscle are complexed with proteins that mediate signals from SNS, including β-adrenergic receptors and β-arrestin (Mongillo et al., 2004, Perry et al., 2002 and Xiang et al., 2005).

The PDE superfamily is subgrouped into 11 families that include at least 20 genes and 50 unique isoforms. Of these PDE families, only PDE4, PDE7, and PDE8 are cAMP specific (Conti et al., 2003). Through alternative splicing and the use of multiple promotors, the PDE4D gene encodes nine variants (PDE4D1-9) with identical catalytic domains and carboxyl termini and unique amino termini important for subcellular localization. For example, PDE4D3 binds to the targeting protein mAKAP via its unique N-terminal region, creating a mAKAP-PKA-PDE4D3 signaling module (Dodge et al., 2001 and Tasken et al., 2001) in which PKA phosphorylation increases PDE4D3 activity approximately 2-fold (Carlisle Michel et al., 2004 and Sette and Conti, 1996). Moreover, as the inventors have previously shown, mAKAP colocalizes with the ryanodine receptor (RyR2)/calcium-release channel in cardiac muscle (Ruehr et al., 2003 and Yang et al., 1998), where it is part of the RyR2 macromolecular signaling complex (Marx et al., 2000 and Wehrens et al., 2003).

PKA-PDE signaling has been identified as a therapeutic target in several major diseases (Conti et al., 2003). Inhibitors of the PDE4 family are under development for asthma, chronic obstructive lung disease (COPD), cognitive disorders including Alzheimer's disease, and stroke (Gong et al., 2004, Gretarsdottir et al., 2003 and Vignola, 2004). However, nonspecific PDE inhibition with theophylline, commonly used to treat asthma and COPD, and trials using PDE3 inhibition to treat heart failure have demonstrated increased mortality due to cardiac arrhythmias (Barnes, 2003 and Packer et al., 1991).

The inventors now, for the first time, definitively demonstrate that PDE4D deficiency in mice is associated with a cardiac phenotype comprised of a progressive, age-related cardiomyopathy and exercise-induced arrhythmias, despite normal global cAMP signaling. Furthermore, PDE4D3 was found to be an integral component of the RyR2 macromolecular signaling complex. RyR2 located on the sarcoplasmic reticulum (SR) is the major Ca²⁺-release channel required for excitation-contraction coupling in heart muscle. RyR2 channels were PKA hyperphosphorylated and exhibited a “leaky” phenotype in PDE4D-deficient mice, similar to RyR2 defects previously observed by the inventors in patients with heart failure and sudden cardiac death (SCD) (Marx et al., 2000 and Wehrens et al., 2003). In failing human hearts, PDE4D3 levels were reduced in the RyR2 complex. Moreover, mice with PDE4D deficiency exhibited accelerated progression of heart failure following myocardial infarction associated with RyR2 channels that were PKA hyperphosphorylated and exhibited a “leaky” phenotype. Pharmacological PDE4 inhibition was associated with exercise-induced cardiac arrhythmias that were suppressed in mice harboring a mutation that prevents PKA phosphorylation of the RyR2 channel. These data indicate that PDE4D deficiency contributes to heart failure and arrhythmias by promoting defective regulation of the RyR2 channel.

### SUMMARY OF THE INVENTION

In view of the foregoing, there is a current need to identify new methods effective for treating and preventing disorders and diseases associated with the RyR recpetors that regulate calcium channel functioning in cells, including skeletal muscular disorders and diseases and especially cardiac disorders and diseases.

The inventors now show, for the first time that that PDE4D plays a protective role in the heart against heart failure and arrhythmias. The inventors also demonstrate herein that PDE deficiency is associated with a severe cardiac phenotype consisting of heart failure and lethal cardiac arrhythmias. Further, the inventors show herein that PDE4D3 deficiency in the RyR2 complex contributes to PKA hypersphosporylation of RyR2 in human and animal hearts, and that PDE4D3 activity provides an important negative feedback mechanism to limit β-AR-dependent PKA phosphorylation of RyR2-Ser2808.

Accordingly, the present invention generally provides compositions useful for treating or preventing a ryanodine receptor associated disorder comprising: a phosphodiesterase (PDE)-associated agent; and optionally a pharmaceutically acceptable carrier. In a preferred embodiment of the invention, the PDE-associated agent may be a PDE protein, a nucleic acid encoding a PDE protein, a member of a PDE signal-transduction pathway, and a modulator of a member of a PDE signal transduction pathway. In one embodiment of the present invention, the PDE-associated agent is PDE4D protein or a nucleic acid encoding a PDE4D protein. The ryanodine receptor associated disorder may be a RyR2-1, RyR2 or RyR3 associated disorder.

The present invention also provides kits for use in delivering a PDE-associated agent to a ryanodine receptor complex in a subject, comprising a PDE-associated agent, optionally with a pharmaceutically acceptable carrier and a catheter.

Additionally, the invention provides methods for treating or preventing a ryanodine receptor associated disorder in a subject comprising augmenting PDE in a ryanodine receptor complex of the subject. In one embodiment of the invention, PDE is augmented in the ryanodine receptor complex by contacting the ryanodine receptor complex with a PDE-associated agent. In one embodiment of the invention, the PDE-associated agent is selected from the group consisting of a PDE protein, a nucleic acid encoding a PDE, a member of a PDE signal-transduction pathway, and a modulator of a member of a PDE signal transduction pathway. In a particular embodiment, the nucleic acid is operatively linked to an inducible promoter. In another embodiment of the present invention, the phosphodiesterase is PDE4D including PDE4D3. The subject of the present invention may be any animal, including amphibians, birds, fish mammals, and marsupials, but is preferably a mammal, including but not necessarily limited to a mouse, rat, cat, dog, horse, monkey cow or pig. In an preferred embodiment, the subject is human.

In a particular embodiment of the invention, the ryanodine receptor associated disorder is a RyR1 receptor associated disorder. In another embodiment of the present invention, the ryanodine receptor associated disorder is a RyR2 receptor associated disorder. In a further embodiment, the ryanodine receptor associated disorder is an RyR3 receptor associated disorder.

In one embodiment of the present invention, the ryanodine receptor associated disorder may be a cardiac disorder and/or disease including, but not limited to, an irregular heartbeat disorder or disease; exercise-induced irregular heart beat disorder or disease; sudden cardiac death; exercise-induced sudden cardiac death; congestive heart failure; chronic obstructive pulmonary disease; and high blood pressure. The irregular heartbeat disorders and diseases and exercise-induced irregular heartbeat disorders and diseases may include, but are not necessarily limited to, atrial and ventricular arrhythmia; atrial and ventricular fibrillation; atrial and ventricular tachyarrhythmia; atrial and ventricular tachycardia; catechlaminergic polymorphic ventricular tachycardia (CPTV); and exercise-induced variants thereof.

In another embodiment of the present invention, the ryanodine receptor associated disorder is a skeletal muscular disorder and/or disease, including but not limited to skeletal muscle fatigue, exercise-induced skeletal muscle fatigue, muscular dystrophy, bladder disorders, and incontinence.

In an additional embodiment of the present invention, the ryanodine receptor associated disorder is a cognitive disorder and/or disease including, but not necessarily limited to Alzheimer's Disease, dementia, forms of memory loss, and age-dependent memory loss. In a further embodiment of the present invention the ryanodine receptor associated disorder is a malignant hyperthermia, diabetes, or sudden infant death syndrome.

The present invention additionally provides methods for regulating PKA phosphorylation of a ryanodine receptor comprising contacting the ryanodine receptor complex with an agent that modulates the level of PDE in the ryanodine receptor complex, wherein contacting the ryanodine receptor complex with an agent that increases the level of PDE in the complex results in a reduction of PKA phosporylation of the ryanodine receptor, and contacting the ryanodine receptor complex with an agent that decreases the level of PDE in the complex results in an increase of PKA phosporylation of the ryanodine receptor. In one embodiment of the invention, the agent is selected from the group consisting of a PDE protein, a nucleic acid encoding a PDE, a member of a PDE signal-transduction pathway, and a modulator of a member of a PDE signal transduction pathway. In another embodiment of the present invention, the phosphodiesterase is PDE4D or PDE4D3.

In a particular embodiment of the invention, the ryanodine receptor is a RyR1 receptor. In another embodiment of the present invention, the ryanodine receptor is a RyR2 receptor. In a further embodiment, the ryanodine receptor is an RyR3 receptor.

Also provided by the present invention are methods for decreasing PKA phosphorylation of a ryanodine receptor by contacting the ryanodine receptor complex with an agent that increases the level of PDE in the ryanodine receptor complex. In one embodiment, the receptor is hyperphosphorylated prior to contacting the ryanodine receptor complex with the agent. In another embodiment, the agent is a PDE protein, a nucleic acid encoding a PDE, a member of a PDE signal-transduction pathway, and a modulator of a member of a PDE signal transduction pathway. In another embodiment of the present invention, the phosphodiesterase is PDE4D or PDE4D3.

In one embodiment of the invention, the ryanodine receptor is a RyR1 receptor. In another embodiment, the ryanodine receptor is a RyR2 associated receptor. In a further embodiment, the ryanodine receptor is a RyR3 associated receptor.

The present invention additionally provides methods for regulating Ca²⁺ release and reuptake in the sarcoplasmic reticulum of a cell comprising contacting a ryanodine receptor complex of the cell with an agent that modulates the level of PDE, wherein contacting the ryanodine receptor complex with an agent that increases the level of PDE results in a reduction of Ca²⁺ release from and reuptake into the sarcoplasmic reticulum and contacting the ryanodine receptor complex with an agent that decreases the level of PDE in the complex results in an increase of Ca²⁺ release from and reuptake into the sarcoplasmic reticulum.

In one embodiment of the invention, the agent is selected from the group consisting of a PDE protein, a nucleic acid encoding a PDE, a member of a PDE signal-transduction pathway, and a modulator of a member of a PDE signal transduction pathway. In another embodiment of the present invention, the phosphodiesterase is PDE4D or PDE4D3.

In an embodiment of the invention, the ryanodine receptor is a RyR1 receptor. In another embodiment, the ryanodine receptor is a RyR2 receptor. In a further embodiment, the ryanodine receptor is a RyR3 receptor.

The present invention further provides methods for decreasing Ca²⁺ release and reuptake in the sarcoplasmic reticulum of a cell comprising contacting a ryanodine receptor complex of the cell with an agent that increases the level of PDE. In one embodiment, the receptor is hyperphosphorylated prior to contacting the ryanodine receptor complex with the agent. In another embodiment, the agent is selected from the group consisting of a PDE protein, a nucleic acid encoding a PDE, a member of a PDE signal-transduction pathway, and a modulator of a member of a PDE signal transduction pathway. In a further embodiment, the phosphodiesterase is PDE4D, including PDE4D3.

In an embodiment of the invention, the ryanodine receptor is a RyR1 receptor. In another embodiment, the ryanodine receptor is a RyR2 receptor. In a further embodiment, the ryanodine receptor is a RyR3 receptor.

### BRIEF DESCRIPTION OF THE FIGURES

FIG. 1 PDE4D Deficiency Promotes Age-Related Cardiomyopathy. *p<0.05 versus wt; #p<0.05 versus PDE4D^{-/-}. (A) Echocardiography at 3 month intervals showing progressive increase in left ventricular end-diastolic diameter (LVEDD) in PDE4D^{-/-} mice (open squares, wt; filled squares, PDE4D^{-/-}; n=12 each). Data in (A)-(D) are mean ±SEM. (B) Age-dependent increase in heart-to-body-weight ratio (HW/BW) in PDE4D^{-/-} mice (open bar, wt; filled bar, PDE4D^{-/-}). (C) Age-dependent decrease in ejection fraction (EF) in PDE4D^{-/-} mice (open bar, wt; filled bar, PDE4D^{-/-}). (D) Reduced cardiac contractility (dP/dt/P_{id}) in PDE4D^{-/-} mice at 3, 9, and 15 months of age (open squares, wt; filled squares, PDE4D^{-/-}). (E) Histology showing dilated cardiomyopathy in PDE4D-deficient mouse hearts.

FIG. 2 Normal cAMP and β-Adrenergic-Receptor Levels in PDE4D^{-/-} Mouse Hearts and Increased cAMP Levels at the Z Lines Detected by FRET-PKA. (A) cAMP concentrations were not significantly increased in hearts of 3- to 6-month-old PDE4D^{-/-} mice (wt, n=5; PDE4D^{-/-}, n=5; p=NS). Each heart was extracted and assayed separately in quadruplicate experiments. Data in (A), (B), and (E) are mean ±SD. (B) β-adrenergic-receptor density was unchanged in PDE4D^{-/-} mice (wt, n=5; PDE4D^{-/-}, n=5; p=NS). (C) Comparison of the Bₘₐₓ values for β-adrenergic-receptor density calculated separately for each of the five wt or five PDE4D^{-/-} knockout mice investigated. (D) FRET-PKA showing increased cAMP-dependent signal over the Z lines (site of localization of RyR2) after low-dose isoproterenol stimulation in PDE4D^{-/-} cardiomyocyte when compared to wild-type cardiomyocyte (white and black areas represent sensing of highest and lowest cAMP concentrations, respectively). (E) Bar graph summarizes Z line intensity-profile analysis of 480 nm/545 nm intensity ratio from five wt and six PDE4D^{-/-} cells (*p<0.05). Dimensions as indicated.

FIG. 3 shows Age-Dependent Alterations in RyR2-Channel Complex and Function in PDE4D^{-/-} Murine Heart. (A) Immunoblots showing progressive increase in PKA phosphorylation of RyR2 at Ser2808 (second panel) but no change in CaMKII phosphorylation at Ser2814 (third panel) and progressive decrease in calstabin2 binding to the RyR2 complex (fourth panel). PKA catalytic (-C) and regulatory (-RII) subunits are not changed during heart failure (HF) development (fifth and six panels). (B) Quantification of age-dependent increase in RyR2 PKA phosphorylation (open bars, wt; filled bars, PDE4D^{-/-}; *p<0.05 versus wt, #p<0.05 versus PDE4D^{-/-}). Data in (B)-(E) and (G) are mean ±SD. (C) Quantification of age-dependent decrease in calstabin2 in the RyR2 complex (open bars, wt; filled bars, PDE4D^{-/-}; *p<0.05 versus wt, #p<0.05 versus PDE4D^{-/-}). (D) Quantification of CaMKII-specific RyR2 phosphorylation showing no significant changes with aging. (E) Immunoprecipitation of RyR2 showing complete absence of PDE activity in the RyR2 complex in PDE4D^{-/-} mouse hearts. *p<0.05 versus wt; #p<0.05 versus untreated wt. Rol, rolipram; Mil, milrinone. (F) Single-channel recordings of wt (left) and PDE4D^{-/-} RyR2 (right) at 3 months of age (top) and 15 months of age (bottom). Current trace from 3-month-old PDE4D^{-/-} heart shows slightly increased channel open probability (Po) with short openings consistent with increased PKA phosphorylation. At 15 months of age, RyR2 channels from PDE4D^{-/-} hearts showed significantly increased Po and subconductance states (indicated by dotted lines), as evidenced by current-amplitude histograms. Upper traces represent 5 s; lower traces represent 500 ms. Channel openings are upward; full openings are 4 pA; closed state is indicated by “c.” (G) Bar graphs summarizing Po, open frequency (Fo), average open time (To), and average closed time (Tc) at 15 months of age in wt and PDE4D^{-/-} groups. *p<0.001; n=10 each.

FIG. 4 PDE4D3 Is a Component of the RyR2 Ca²⁺-Release-Channel Complex. (A) PAN-PDE4 antibody against the conserved UCR2 domain (a-4PAN, top panel) and antibody raised against the N-terminal domain unique to PDE4D3 (α-4D3, bottom panel) were used to detect PDE4D isoforms in extracts of COS7 cells overexpressing recombinant PDE4D splice variants 1 to 9. Samples were size fractionated on 6% SDS-PAGE and blotted onto Immobilon-P membranes. (B) Immunoblotting with splice-variant-specific anti-PDE4D3, anti-PDE4D8, and anti-PDE4D9 antibodies shows expression of all three major forms in the heart. However, immunoprecipitation of RyR2 followed by splice-variant-specific immunoblot demonstrates that only PDE4D3 is associated with RyR2. Reverse immunoprecipitation with a specific anti-PDE4D3 antibody confirms that PDE4D3 is physically associated with RyR2. (C) Immunoblots of cardiac lysates showing amounts of RyR2 and PDE4D3 in wt, PDE4D^{+/-}, and PDE4D^{-/-} mice. Bar graph shows a 37% reduction of PDE4D3 in the RyR2 complex in cardiac lysates of PDE4D^{+/-} mice relative to wt. *p<0.05, n=3 for each genotype. Data in (C) and (D) are mean ±SD. (D) Coimmunoprecipitation using anti-RyR2 antibody, showing a 44% decrease of PDE4D3 bound to RyR2 in PDE4D^{+/-} mouse hearts relative to wt. *p<0.05, n=3 for each genotype.

FIG. 5 Reduced PDE4D3 in the RyR2 Complex in Human Heart Failure. (A) PDE4D3 was detected in human cardiac lysate and in the immunoprecipitated RyR2 complex (IP:RyR2); IP:IgG, negative control. (B) RyR2 was immunoprecipitated from cardiac homogenates of normal human (N) and heart failure (HF) samples. PDE4D3 binding to RyR2 was significantly decreased in human HF. Increased PKA phosphorylation was detected by a phosphoepitope-specific RyR2-Ser2808 antibody in HF samples. (C) RyR2 bound PDE4D3 activity was significantly decreased in HF, as evidenced by close-proximity substrate cAMP catalysis (#p<0.001). Data in (C) and (D) are mean ±SD. (D) Rolipram (R), a PDE4-specific inhibitor, but not milrinone (M), a PDE3-specific inhibitor, decreased RyR2-associated PDE activity submaximally; C, control (#p<0.001 versus untreated sample).

FIG. 6 Cardiac Arrhythmias due to PDE4D3 Inhibition Are Suppressed in Mice Harboring Mutant RyR2 that Cannot be PKA Phosphorylated. (A) Susceptibility to exercise-induced sustained ventricular arrhythmias (sVT) and nonsustained ventricular arrhythmias (nsVT) was significantly increased in PDE4D^{-/-} compared to wt mice (each n=6, *p<0.05). (B) Rolipram (0.3 mg/kg body weight) maximally increased RyR2-Ser2808 PKA phosphorylation during exercise in vivo. Treatment as indicated on top: Rol, rolipram; Epi, epinephrine (0.1 mg/kg); white bars, no rolipram; black bar, rolipram-treated mice; *p<0.05 between treatments in wt mice; #p<0.001 wt versus homozygous RyR2-S2808A knockin (S2808A^{+/+}) mice. Control mice were treated with placebo (rolipram carrier, 0.5% DMSO). Data are mean ±SD. (C) In rolipram-treated mice, susceptibility to exercise-induced sustained ventricular arrhythmias (sVT) and nonsustained ventricular arrhythmias (nsVT) was significantly decreased in homozygous RyR2-S2808A knockin (S2808A^{+/+}) mice compared to wt mice (each n=6, #p<0.05). (D) Mortality from sudden death was significantly increased in PDE4D^{+/-} mice 24 and 72 hr after MI compared to wt mice. White bars, wt mice; black bars, PDE4D^{+/-} mice. *p<0.01 between wt and PDE4D^{+/-} groups.

FIG. 7 PDE4D3 Deficiency Promotes HF Progression. (A) LVEDD increased in PDE4D^{+/-} (black squares) compared to wt (open squares) mice before (control, CO) and 14 and 28 days after myocardial infarction (MI) (*p<0.05 versus wt). Both treatment with the 1,4-benzothiazepine JTV-519 (red line), which enhances calstabin2 binding to RyR2, or crossing the PDE4D^{+/-} mice with RyR2-S2808A mice that harbor RyR2 that cannot be PKA phosphorylated (green line) significantly reduced the remodeling of the left ventricle following MI (#p<0.01 versus PDE4D^{+/-}). Data in (A)-(C) are mean ±SEM. (B) Reduced cardiac EF in PDE4D^{+/-} mice 28 days after MI. wt, open bars; PDE4D^{+/-} mice, filled bars (*p<0.05 versus CO same genotype). Both treatment with the 1,4-benzothiazepine JTV-519 (red bar), which enhances calstabin2 binding to RyR2, or crossing the PDE4D^{+/-} mice with RyR2-S2808A mice that harbor RyR2 that cannot be PKA phosphorylated (green bar) significantly improved left ventricular EF following MI (#p<0.01 versus PDE4D^{+/-} MI). (C) Left ventricular contractility (dP/dt)/P_{id} normalized to 100% of control in wt (open bars) and haploinsufficient PDE4D^{+/-} mice (black bars) before (control, CO) and 28 days after MI (*p<0.05 versus CO in same genotype). Treatment with JTV-519 (red bar) or crossing the PDE4D^{+/-} mice with RyR2-S2808A mice (green bar) significantly improved contractility following MI (#p<0.01 versus PDE4D^{+/-} MI). (D) Immunoblot showing levels of PDE4D3, calstabin2, and RyR2-Ser2808 PKA phosphorylation in the immunoprecipitated RyR2-channel complex. JTV-519 treatment (red) or crossing the PDE4D^{+/-} mice with RyR2-S2808A mice (green) significantly increased the binding of calstabin2 to RyR2 in the PDE4D-deficient mice. (E) Quantification of RyR2 PKA phosphorylation in wt and PDE4D^{+/-} mice before (CO) and 28 days after MI, showing significantly more RyR2 PKA hyperphosphorylation in PDE4D^{+/-} mice (*p<0.05 versus wt at same time point). Treatment with JTV-519 (red bar) or crossing the PDE4D^{+/-} mice with RyR2-S2808A mice (green bar) significantly reduced RyR2 PKA phosphorylation following MI (#p<0.01 versus PDE4D^{+/-} MI). Data in (E) and (F) are mean ±SD. (F) Quantification of RyR2-associated PDE activity showing significant decrease in PDE4D^{+/-} mice 28 days before (CO) and after MI compared to wt (*p<0.05 versus wt). Treatment with JTV-519 (red bar) or crossing the PDE4D^{+/-} mice with RyR2-S2808A mice (green bar) significantly increased RyR2-associated PDE activity following MI (#p<0.01 versus PDE4D^{+/-} MI).

### DETAILED DESCRIPTION OF THE INVENTION

As used herein and in the appended claims the singular forms “a,” “an,” and “the” include plural references unless the content clearly dictates otherwise. Thus, for example, reference to “an agent” includes a plurality of such agents and equivalents thereof known to those skilled in the art, and so forth. All publications, patent applications, patents and other references mentioned herein are incorporated by reference in there entirety.

The inventors have previously shown in co-pending U.S. application Ser. No. 10/7894,218 (incorporated in its entirety herein) that PDE4D3 is present in the RyR1 receptor complex of skeletal muscle. Specifically, to determine whether mAKAP and PDE4D3 were present in the RyR1 macromolecular complex, immunoprecipitation with anti-RyR-5029 followed by immunoblotting with antibodies that detect mAKAP and PDE4D3 was used. The inventors found that both mAKAP and PDE4D3 co-immunoprecipitate with RyR1 and therefore are part of the RyR1 macromolecular complex. Moreover, the inventors found that compared to controls, the amount of PDE4D3 in the RyR1 channel complex could contribute to a local increase in cAMP and increased PKA activity that likely explains PKA hyperphosphorylation of RyR1 in HF skelatal muscle.

The inventors have also previously posited in co-pending U.S. application Ser. No. 10/608,723 (incorporated in its entirety herein) that a chemical agent that reduces PKA phosphorylation of a RyR2 receptor could at via multifarious mechanisms, including, inter alia, inhibiting PKA activity, or increasing the activity of endogenous phosphatases (PP1 and PP2A have been shown to be present in the RyR2 macromolecular complex), or increasing the activity of a phosphodiesterase which hydrolyzes cAMP (PDE4D3, which hydrolyzes cAMP, has also been shown to be present in the RyR2 macromolecular complex; Dodge et al., 2001).

The inventors demonstrate herein a novel function of PDE4D3 in the regulation of intracellular Ca²⁺ release. PDE4D3 activity provides an important negative feedback mechanism to limit βPAR-dependent PKA phosphorylation of RyR2-Ser²⁸⁰⁹. Under physiologic conditions, PDE4D3 regulates local PKA activity and channel activation at RyR2-Ser²⁸⁰⁹ and prevents excess accumulation of cAMP and uncontrolled PKA activation. In human heart failure, PDE4D3 deficiency contributes to RyR2 PKA hyperphosphorylation, calstabin2 (FKBP12.6) depletion and hyperactive, “leaky” RyR2 channels. Since global cAMP synthesis is decreased in HF (Feldman, 1987), a reduced capacity for cAMP hydrolysis by locally reduced PDE4D3 activity may be causative for RyR2 PKA hyperphosphorylation. Taken together, these data suggest that PDE4D3 plays a protective role in the heart against HF and catecholaminergic arrhythmias. On the other hand, chronic pharmacologic inhibition of the PDE4 class of enzymes may contribute to a cardiac phenotype, particularly in individuals with underlying cardiac disease. In addition there are likely to be other signaling systems affected by reduced PDE4 activity, since β-arrestin targeting of PDE4D3 activity may be important for p2AR desensitization²⁸ and PDE4D ablation differentially changes βIAR versus β2AR signaling. Importantly, PDE4D3 deficiency was associated with stress-induced cardiac arrhythmias indicating a possible novel mechanism for drug-induced sudden death analogous to the drug-induced long QT syndrome caused by blockage of HERG potassium channels, suggesting that PDE4 inhibitors could increase the risk of sudden cardiac death via an RyR2-mediated mechanism.

In veiw of the foregoing, the present invention provides novel compounds and methods for treating and preventing disorders and diseases associated with the RyR receptors that regulate calcium channel functioning in cells.

Specifically, the present invention encompasses and provides compositions useful for treating or preventing a ryanodine receptor associated disorder comprising a phosphodiesterase (PDE)-associated agent; and optionally a pharmaceutically acceptable carrier. In a preferred embodiment of the invention, the PDE-associated agent may be a PDE protein, a nucleic acid encoding a PDE protein, a member of a PDE signal-transduction pathway, and a modulator of a member of a PDE signal transduction pathway. In one embodiment of the present invention, the PDE-associated agent is PDE4D protein or a nucleic acid encoding a PDE4D protein. The ryanodine receptor associated disorder may be a RyR2-1, RyR2 or RyR3 associated disorder.

As used herein the term “ryanodine receptor associated disorders” and “disorders and diseases associated with the RyR receptors” means disorders and diseases that can be treated and/or prevented by modulating PDE in the RyR receptor complex in cells. “Ryanodine receptor associated disorders” and/or “Disorders and diseases associated with the RyR receptors” include, without limitation, cardiac disorders and diseases, skeletal muscular disorders and diseases, cognitive disorders and diseases, malignant hyperthermia, diabetes, and sudden infant death syndrome. Cardiac disorder and diseases include, but are not limited to, irregular heartbeat disorders and diseases; exercise-induced irregular heartbeat disorders and diseases; sudden cardiac death; exercise-induced sudden cardiac death; congestive heart failure; chronic obstructive pulmonary disease; and high blood pressure. Irregular heartbeat disorders and diseases include and exercise-induced irregular heartbeat disorders and diseases include, but are not limited to, atrial and ventricular arrhythmia; atrial and ventricular fibrillation; atrial and ventricular tachyarrhythmia; atrial and ventricular tachycardia; catecholaminergic polymorphic ventricular tachycardia (CPVT); and exercise-induced variants thereof. Skeletal muscular disorder and diseases include, but are not limited to, skeletal muscle fatigue, exercise-induced skeletal muscle fatigue, muscular dystrophy, bladder disorders, and incontinence. Cognitive disorders and diseases include, but are not limited to, Alzheimer's Disease, forms of memory loss, and age-dependent memory loss.

As used herein, “RyR” includes RyR1, RyR2, and RyR3, and also includes an “RyR protein” and an “RyR analogue.” An “RyR analogue” is a functional variant of the RyR protein, having RyR biological activity, that has 60% or greater amino-acid-sequence homology with the RyR protein. The RyR of the present invention are unphosphorylated, phosphorylated (e.g., by PKA), or hyperphosphorylated (e.g., by PKA). As further used herein, the term “RyR biological activity” refers to the activity of a protein or peptide that demonstrates an ability to associate physically with, or bind with, FKBP12 (calstabin1) in the case of RyR1 and RyR3, and FKBP12.6 (calstabin2) in the case of RyR2 (i.e., binding of approximately two fold or, approximately five fold, above the background binding of a negative control), under the conditions of the assays described herein.

As used herein, “ryanodine receptor complex” or “RyR complex” refers to the RyR macromolecular signaling complex as described herein.

Additionally, the present invention provides methods for treating or preventing a ryanodine receptor associated disorder in a subject comprising augmenting PDE in a ryanodine receptor complex of the subject. In one embodiment of the invention, PDE is augmented in the ryanodine receptor complex by contacting the ryanodine receptor complex with a PDE-associated agent. In one embodiment of the invention, the PDE-associated agent is selected from the group consisting of a PDE protein, a nucleic acid encoding a PDE, a member of a PDE signal-transduction pathway, and a modulator of a member of a PDE signal transduction pathway. In a particular embodiment, the nucleic acid is operatively linked to an inducible promoter. In another embodiment of the present invention, the phosphodiesterase is PDE4D including PDE4D3. The subject of the present invention may be an in vitro or in vivo system and any animal, including amphibians, birds, fish mammals, and marsupials, but is preferably a mammal, including but not necessarily limited to a mouse, rat, cat, dog, horse, monkey cow or pig. In a preferred embodiment of the invention, the subject is human.

In a particular embodiment of the invention, the ryanodine receptor associated disorder is a RyR1 receptor associated disorder. In another embodiment of the present invention, the ryanodine receptor associated disorder is a RyR2 receptor associated disorder. In a further embodiment, the ryanodine receptor associated disorder is an RyR3 receptor associated disorder.

The cells of a subject include striated muscle cells. A striated muscle is a muscle in which the repeating units (sarcomeres) of the contractile myofibrils are arranged in registry throughout the cell, resulting in transverse or oblique striations that are observed at the level of a light microscope. Examples of striated muscle cells include, without limitation, voluntary (skeletal) muscle cells and cardiac muscle cells. In one embodiment, the cell used in the method of the present invention is a human cardiac muscle cell. As used herein, the term “cardiac muscle cell” includes cardiac muscle fibers, such as those found in the myocardium of the heart. Cardiac muscle fibers are composed of chains of contiguous heart-muscle cells, or cardiomyocytes, joined end to end at intercalated disks. These disks possess two kinds of cell junctions: expanded desmosomes extending along their transverse portions, and gap junctions, the largest of which lie along their longitudinal portions.

In one embodiment of the present invention, the ryanodine receptor associated disorder may be a cardiac disorder and/or disease including, but not limited to, an irregular heartbeat disorder or disease; exercise-induced irregular heart beat disorder or disease; sudden cardiac death; exercise-induced sudden cardiac death; congestive heart failure; chronic obstructive pulmonary disease; and high blood pressure. The irregular heartbeat disorders and diseases and exercise-induced irregular heartbeat disorders and diseases may include, but are not necessarily limited to, atrial and ventricular arrhythmia; atrial and ventricular fibrillation; atrial and ventricular tachyarrhythmia; atrial and ventricular tachycardia; catechlaminergic polymorphic ventricular tachycardia (CPTV); and exercise-induced variants thereof.

In another embodiment of the present invention, the ryanodine receptor associated disorder is a skeletal muscular disorder and/or disease, including but not limited to skeletal muscle fatigue, exercise-induced skeletal muscle fatigue, muscular dystrophy, bladder disorders, and incontinence.

In an additional embodiment of the present invention, the ryanodine receptor associated disorder is a cognitive disorder and/or disease including, but not necessarily limited to Alzheimer's Disease, dementia, forms of memory loss, and age-dependent memory loss. In a further embodiment of the present invention the ryanodine receptor associated disorder is a malignant hyperthermia, diabetes, or sudden infant death syndrome.

As used herein, “effective amount” or “pharmaceutically effective amount” refers to any amount of an agent which, when administered to a subject suffering from a disorder against which the agent is effective, causes reduction, remission or regression or prevents recurrence of the disorder. “Prophylactically effective amount” refers to any amount of an agent which, when administered to a subject prone to suffer from a disorder, inhibits the onset of the disorder.

The present invention additionally provides methods for regulating PKA phosphorylation of a ryanodine receptor comprising contacting the ryanodine receptor complex with an agent that modulates the level of PDE in the ryanodine receptor complex, wherein contacting the ryanodine receptor complex with an agent that increases the level of PDE in the complex results in a reduction of PKA phosporylation of the ryanodine receptor, and contacting the ryanodine receptor complex with an agent that decreases the level of PDE in the complex results in an increase of PKA phosporylation of the ryanodine receptor. In one embodiment of the invention, the agent is selected from the group consisting of a PDE protein, a nucleic acid encoding a PDE, a member of a PDE signal-transduction pathway, and a modulator of a member of a PDE signal transduction pathway. In preferred embodiment of the present invention, the phosphodiesterase is PDE4D or PDE4D3.

In a particular embodiment of the invention, the ryanodine receptor is a RyR1 receptor. In another embodiment of the present invention, the ryanodine receptor is a RyR2 receptor. In a further embodiment, the ryanodine receptor is an RyR3 receptor.

As used herein, “PKA phosphorylation” refers to a reaction in which a phosphate group is substituted for a hydroxyl group by the enzyme protein kinase A (PKA).

The present invention also provides kits for use in delivering a PDE-associated agent to a ryanodine receptor complex in a subject, comprising a PDE-associated agent, optionally with a pharmaceutically acceptable carrier and a catheter.

Also provided by the present invention are methods for decreasing PKA phosphorylation of a ryanodine receptor by contacting the ryanodine receptor complex with an agent that increases the level of PDE in the ryanodine receptor complex. In one embodiment, the receptor is hyperphosphorylated prior to contacting the ryanodine receptor complex with the agent. In another embodiment, the agent is a PDE protein, a nucleic acid encoding a PDE, a member of a PDE signal-transduction pathway, and a modulator of a member of a PDE signal transduction pathway. In another embodiment of the present invention, the phosphodiesterase is PDE4D or PDE4D3.

In one embodiment of the invention, the ryanodine receptor is a RyR1 receptor. In another embodiment , the ryanodine receptor is a RyR2 associated receptor. In a further embodiment, the ryanodine receptor is a RyR3 associated receptor.

The present invention additionally provides methods for regulating Ca²⁺ release and reuptake in the sarcoplasmic reticulum of a cell comprising contacting a ryanodine receptor complex of the cell with an agent that modulates the level of PDE, wherein contacting the ryanodine receptor complex with an agent that increases the level of PDE results in a reduction of Ca²⁺ release from and reuptake into the sarcoplasmic reticulum and contacting the ryanodine receptor complex with an agent that decreases the level of PDE in the complex results in an increase of Ca²⁺ release from and reuptake into the sarcoplasmic reticulum.

In one embodiment of the invention, the agent is selected from the group consisting of a PDE protein, a nucleic acid encoding a PDE, a member of a PDE signal-transduction pathway, and a modulator of a member of a PDE signal transduction pathway. In a preferred embodiment of the present invention, the phosphodiesterase is PDE4D or PDE4D3.

In an embodiment of the invention, the ryanodine receptor is a RyR1 receptor. In another embodiment, the ryanodine receptor is a RyR2 receptor. In a further embodiment, the ryanodine receptor is a RyR3 receptor.

The present invention further provides methods for decreasing Ca²⁺ release and reuptake in the sarcoplasmic reticulum of a cell comprising contacting a ryanodine receptor complex of the cell with an agent that increases the level of PDE. In one embodiment, the receptor is hyperphosphorylated prior to contacting the ryanodine receptor complex with the agent. In another embodiment, the agent is selected from the group consisting of a PDE protein, a nucleic acid encoding a PDE, a member of a PDE signal-transduction pathway, and a modulator of a member of a PDE signal transduction pathway. In a further embodiment, the phosphodiesterase is PDE4D, including PDE4D3.

In an embodiment of the invention, the ryanodine receptor is a RyR1 receptor. In another embodiment, the ryanodine receptor is a RyR2 receptor. In a further embodiment, the ryanodine receptor is a RyR3 receptor.

PDEs are enzyme proteins, found in certain cells, which hydrolyze phosphodiester bonds. PDEs regulate the local concentration of 3′, 5′ cyclic adenosine monophosphate (cAMP) within cells. In the heart, PDE4 contributes to the regulation of cAMP levels in cardiac myocytes. The PDE superfamily is subgrouped into 11 families tht include at least 20 genes and 50 unique isoforms. The PDE4D gene encodes nnine variants (PDE4D1-9) with identical catalytic domains and carboxyl termini and unique amino termini important for subcellular localization. In a preferred embodiment of the present invention, the PDE is PDE4D3.

As used herein, “PDE” includes both a “PDE protein” and a “PDE analogue”. Unless otherwise indicated, “protein” shall include a protein, protein domain, polypeptide, or peptide, and any fragment or variant thereof having protein function. The variants preferably have greater than about 75% homology with the naturally-occurring protein sequence, more preferably have greater than about 80% homology, even more preferably have greater than about 85% homology, and most preferably, have greater than about 90% homology with the protein sequence. In some embodiments, the homology may be as high as about 93-95%, 98%, or 99%. These variants may be substitutional, insertional, or deletional variants. The variants may also be chemically-modified derivatives: proteins which have been subjected to chemical modification, but which retain the biological characteristics of the naturally-occurring protein.

A “PDE analogue”, as used herein, is a functional variant of the PDE protein, having PDE biological activity, that has 60% or greater (preferably, 70% or greater) amino-acid-sequence homology with the PDE protein. As further used herein, the term “PDE biological activity” refers to the activity of a protein or peptide that demonstrates an ability to hydrolyze cAMP, as described herein.

In accordance with methods described herein, PDE may be augmented or increased in a cell or subcellular compartment, or more particularly, in a ryanodine receptor complex (RyR complex) of a cell by activating, facilitating, inducing, or stimulating one or more functions, activities, or effects (e.g., downstream effects of the PDE in the PDE signal transduction pathway) of PDE in a cell or in a RyR of a cell, particularly those that result in promotion of heart-tissue generation, or by increasing the amount, expression, or level of PDE in the cells. Furthermore, one or more PDE functions, activities, effects, expression, and levels in a cell, subcellular compartment or RyR complex may be augmented by targeting PDE directly, or by targeting PDE indirectly, via an enzyme or other endogenous molecule that regulates or modulates the functions, activities, effects, expression, and/or levels of PDE in the cell. PDE expression may also be augmented by engineering the PDE gene so that PDE is expressed on an inducible promoter. In such a case, PDE expression would be sustained in the presence of a suitable inducing agent, but would shut down once the supply of inducer was depleted, thereby bringing about a decrease in the amount or level of PDE in the cell. PDE also may be augmented in a cell or RyR complex by activating, facilitating, inducing, or stimulating the functions, activities, effects, expression, and levels of endogenous PDE, or by introduction of an exogenous PDE, particularly where the PDE is under the control of a strong promoter.

The functions, activities, effects, expression, and/or levels of PDE are augmented in a cells or RyR complex by an amount effective to promote hydrolyzation of cAMP. This amount may be readily determined by the skilled artisan, based upon known procedures, including analysis of titration curves established in vivo, methods disclosed herein, and techniques known to one of skill in the art.

In the method of the present invention, the functions, activities, effects, expression, and/or levels of PDE in a cell, subcellular compartment or RyR complex are preferably augmented by contacting the cells (i.e., treating the cells) with a PDE-associated agent. As used herein, an “agent” shall include a protein, polypeptide, peptide, nucleic acid (including DNA, RNA, and an antisense oligonucleotide), antibody (monoclonal and polyclonal), Fab fragment, F(ab′)2 fragment, molecule, compound, antibiotic, drug, and any combinations thereof, and may be an agent reactive with PDE or a member of a PDE signal transduction pathway. The term “reactive”, as used herein, means that the molecule or mimetic has affinity for, binds to, or is directed against PDE or a member of a PDE signal transduction pathway. A Fab fragment is a univalent antigen-binding fragment of an antibody, which is produced by papain digestion. A F(ab′)2 fragment is a divalent antigen-binding fragment of an antibody, which is produced by pepsin digestion.

As further used herein, the term “PDE-associated agent” or “agent” includes a PDE protein, including an exogenous PDE protein; a PDE nucleic acid (i, e., a nucleic acid encoding a PDE); a member of a PDE signal-transduction pathway (including upstream and downstream effectors and activators, in either protein or nucleic acid form); and a modulator (e.g., inhibitor, activator, antagonist, or agonist) of a member of the PDE signal transduction pathway or system (i.e., a modulator which affects the expression, activity, function, and/or effect of a member of the PDE signal-transduction pathway), in either protein or nucleic acid form, including a modulator of PDE expression. Additionally, as used herein, a “member of a PDE signal-transduction pathway” includes a downstream effector or an upstream regulator of PDE in cells.

By way of example, activity of PDE in a cell, subcellular compartment or RyR complex may be augmented by contacting the cells or RyR complex with a small molecule or protein mimetic that stimulates PDE activity and/or that is reactive with PDE or a member of a PDE signal transduction pathway. Similarly, the level of PDE in a cell or RyR complex may be augmented by directly or indirectly causing, inducing, or stimulating the upregulation of PDE expression within a subject. Accordingly, in one embodiment of the present invention, activity of PDE is increased in a subject by administering to the subject a modulator of PDE expression.

In one embodiment of the present invention, the PDE-associated agent is a protein. Examples of proteins for use in the present invention include, without limitation, PDE proteins, members of the PDE signal-transduction pathway (including upstream and downstream effector and activator polypeptides), modulators (e.g., inhibitors, activators, antagonists, or agonists) of a member of the PDE signal-transduction pathway/system, PDE-associated antibodies (e.g., IgA, IgD, IgE, IgG, IgM, single-chain antibodies, and Fab′ fragments, such as scFv) that are capable of binding and inhibiting a negative regulator of the PDE signal-transduction pathway, and PDE-associated ligands (e.g., a ligand for a member of the PDE signal-transduction pathway, and derivatives thereof). Preferably, the PDE-associated protein is PDE4D protein.

Where the protein of the present invention is an antibody, the protein is preferably a mammalian antibody (e.g., a human antibody) or a chimeric antibody (e.g., a humanized antibody). More preferably, the antibody is a human or humanized antibody. As used herein, the term “humanized antibody” refers to a genetically-engineered antibody in which the minimum portion of an animal antibody (e.g., an antibody of a mouse, rat, pig, goat, or chicken) that is generally essential for its specific functions is “fused” onto a human antibody. In general, a humanized antibody is 1-25%, preferably 5-10%, animal; the remainder is human. Humanized antibodies usually initiate minimal or no response in the human immune system. Methods for expressing fully human or humanized antibodies in organisms other than human are well known in the art (see e.g., U.S. Pat. No. 6,150,584, Human antibodies derived from immunized xenomice; U.S. Pat. No. 6,162,963, Generation of xenogenetic antibodies; and U.S. Pat. No. 6,479,284, Humanized antibody and uses thereof). In one embodiment of the present invention, the antibody is a single-chain antibody. In one embodiment, the single-chain antibody is a human or humanized single-chain antibody. In another embodiment of the present invention, the antibody is a murine antibody.

The PDE-associated agent of the present invention may also be a nucleic acid. As used herein, a “nucleic acid” or “polynucleotide” includes a nucleic acid, an oligonucleotide, a nucleotide, a polynucleotide, and any fragment or variant thereof. The nucleic acid or polynucleotide may be double-stranded, single-stranded, or triple-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, and xanthine hypoxanthine. The nucleic acid or polynucleotide may be combined with a carbohydrate, lipid, protein, or other materials. In one embodiment of the present invention, the nucleic acid encodes PDE4D protein.

The “complement” of a nucleic acid refers, herein, to a nucleic acid molecule which is completely complementary to another nucleic acid, or which will hybridize to the other nucleic acid under conditions of high stringency. High-stringency conditions are known in the art (see e.g., Maniatis et al., Molecular Cloning: A Laboratory Manual, 2nd ed. (Cold Spring Harbor: Cold Spring Harbor Laboratory, 1989) and Ausubel et al., eds., Current Protocols in Molecular Biology (New York, N.Y.: John Wiley & Sons, Inc., 2001)). Stringent conditions are sequence-dependent, and may vary depending upon the circumstances. As used herein, the term “cDNA” refers to an isolated DNA polynucleotide or nucleic acid molecule, or any fragment, derivative, or complement thereof. It may be double-stranded, single-stranded, or triple-stranded, it may have originated recombinantly or synthetically, and it may represent coding and/or noncoding 5′ and/or 3′ sequences.

The nucleic acid agent of the present invention, for example, may be a plasmid. Such a plasmid may comprise a nucleic acid sequence encoding PDE or another PDE-associated protein, although it is to be understood that other types of nucleic acid agents, such as recombinant viral vectors, may also be used for the purposes of the present invention. In one embodiment of the present invention, the nucleic acid (e.g., plasmid) encodes at least one PDE-associated protein. In a preferred embodiment, the nucleic acid encodes PDE4D protein.

The term “plasmid”, as used herein, refers generally to circular double-stranded DNA, which is not bound to a chromosome. The DNA, for example, may be a chromosomal or episomal-derived plasmid. The plasmid of the present invention may optionally contain a terminator of transcription, a promoter, and/or a discrete series of restriction-endonuclease recognition sites, located between the promoter and the terminator. In the plasmid, a polynucleotide insert of interest (e.g., one encoding a PDE-associated protein) should be operatively linked to an appropriate promoter. The promoter may be its native promoter or a host-derived promoter. The promoter may also be a tissue-specific promoter, such as a cardiomyocyte-specific promoter or other heart-tissue-specific promoter. The promoter may further be a regulatable promoter, which may be turned off when the expression of the gene is no longer desired. Examples of promoters for use in the present invention include the actin promoter and viral promoters. Other suitable promoters will be known to the skilled artisan.

In another embodiment of the present invention, the nucleic acid (e.g., plasmid) encodes or comprises at least one gene-silencing cassette, wherein the cassette is capable of silencing the expression of genes that negatively affect the PDE signal-transduction pathway/system. It is well understood in the art that a gene may be silenced at a number of stages, including, without limitation, pre-transcription silencing, transcription silencing, translation silencing, post-transcription silencing, and post-translation silencing. In one embodiment of the present invention, the gene-silencing cassette encodes or comprises a post-transcription gene-silencing composition, such as antisense RNA or RNAi. Both antisense RNA and RNAi may be produced in vitro, in vivo, ex vivo, or in situ.

For example, the PDE-associated agent of the present invention may be an antisense RNA. Antisense RNA is an RNA molecule with a sequence complementary to a specific RNA transcript, or mRNA, whose binding prevents further processing of the transcript or translation of the mRNA. Antisense molecules may be generated, synthetically or recombinantly, with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are sensitive to nucleases. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein et al., Oligodeoxynucleotides as inhibitors of gene expression: a review, Cancer Res., 48:2659-68, 1998).

Antisense molecules designed to bind to the entire mRNA may be made by inserting cDNA into an expression plasmid in the opposite or antisense orientation. Antisense molecules may also function by preventing translation initiation factors from binding near the 5′ cap site of the mRNA, or by interfering with interaction of the mRNA and ribosomes (see e.g., U.S. Pat. No. 6,448,080, Antisense modulation of WRN expression; U.S. Patent Application No. 2003/0018993, Methods of gene silencing using inverted repeat sequences; U.S. Patent Application No. 2003/0017549, Methods and compositions for expressing polynucleotides specifically in smooth muscle cells in vivo; Tavian et al., Stable expression of antisense urokinase mRNA inhibits the proliferation and invasion of human hepatocellular carcinoma cells, Cancer Gene Ther., 10:112-20, 2003; Maxwell and Rivera, Proline oxidase induces apoptosis in tumor cells and its expression is absent or reduced in renal carcinoma, J. Biol. Chem., 278:9784-89, 2003; Ghosh et al., Role of superoxide dismutase in survival of Leishmania within the macrophage, Biochem. J., 369:447-52, 2003; and Zhang et al., An anti-sense construct of full-length ATM cDNA imposes a radiosensitive phenotype on normal cells, Oncogene, 17:811-8, 1998).

Oligonucleotides antisense to a member of the PDE signal-transduction pathway/system may be designed based on the nucleotide sequence of the member of interest. For example, a partial sequence of the nucleotide sequence of interest (generally, 15-20 base pairs), or a variation sequence thereof, may be selected for the design of an antisense oligonucleotide. This portion of the nucleotide sequence may be within the 5′ domain. A nucleotide sequence complementary to the selected partial sequence of the gene of interest, or the selected variation sequence, then may be chemically synthesized using one of a variety of techniques known to those skilled in the art, including, without limitation, automated synthesis of oligonucleotides having sequences which correspond to a partial sequence of the nucleotide sequence of interest, or a variation sequence thereof, using commercially-available oligonucleotide synthesizers, such as the Applied Biosystems Model 392 DNA/RNA synthesizer.

Once the desired antisense oligonucleotide has been prepared, its ability to modulate PDE then may be assayed. For example, the antisense oligonucleotide may be contacted with a cells or RyR receptor complex, and the levels of PDE expression or activity in the cells or RyR complex may be determined using standard techniques, such as Western-blot analysis and immunostaining. Alternatively, the antisense oligonucleotide may be delivered to a cell or RyR complex using a liposome vehicle, then the levels of PDE expression or activity in the cells may be determined using standard techniques, such as Western-blot analysis and immunostaining. Where the level of PDE expression in the cells is increased in the presence of the designed antisense oligonucleotide, it may be concluded that the oligonucleotide could be an appropriate PDE-associated agent for use in modulating PDE in cells.

It is within the confines of the present invention that oligonucleotides antisense to a member of the PDE signal-transduction pathway/system may be linked to another agent, such as a drug or a ribozyme, in order to increase the effectiveness of treatments using PDE-associated agents and/or to increase the efficacy of targeting. Moreover, antisense oligonucleotides may be prepared using modified bases (e.g., a phosphorothioate), as discussed above, to make the oligonucleotides more stable and better able to withstand degradation.

The PDE-associated agent of the present invention also may be an interfering RNA, or RNAi, including PDE small interfering RNA (siRNA). As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment, or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

In one embodiment of the present invention, RNAi is produced in vivo by an expression vector containing a gene-silencing cassette coding for RNAi. (see e.g., U.S. Pat. No. 6,278,039, C. elegans deletion mutants; U.S. Patent Application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes). In another embodiment of the present invention, RNAi is produced in vitro, synthetically or recombinantly, and transferred into the microorganism using standard molecular-biology techniques. Methods of making and transferring RNAi are well known in the art (see e.g., Ashrafi et al., Genome-wide RNAi analysis of Caenorhabditis elegans fat regulatory genes, Nature, 421:268-72, 2003; Cottrell et al., Silence of the strands: RNA interference in eukaryotic pathogens, Trends Microbial., 11:37-43, 2003; Nikolaev et al., Parc: A Cytoplasmic Anchor for p53, Cell, 112:29-40, 2003; Wilda et al., Killing of leukemic cells with a BCR/ABL fusion gene RNA interference (RNAi), Oncogene, 21:5716-24, 2002; Escobar et al., RNAi-mediated oncogene silencing confers resistance to crown gall tumorigenesis, Proc. Natl. Acad. Sci. USA, 98:13437-42, 2001; and Billy et al., Specific interference with gene expression induced by long, double-stranded RNA in mouse embryonal teratocarcinoma cell lines, Proc. Natl. Acad. Sci. USA, 98:14428-33, 2001).

In a further embodiment of the present invention, the plasmid is an expression plasmid. The expression plasmid may contain sites for transcription initiation, termination, and, optionally, in the transcribed region, a ribosome-binding site for translation. The coding portions of the mature transcripts expressed by the plasmid may include a translation initiating codon at the beginning, and a termination codon appropriately positioned at the end of the polypeptide to be translated.

By way of example, the PDE-associated gene to be expressed from the expression plasmid may be under the specific regulatory control of certain types of promoters. In one embodiment, these promoters are constitutive promoters. Genes under the control of these constitutive promoters will be expressed continually. In another embodiment, the promoters are inducible promoters. Genes under the control of these inducible promoters will be expressed only upon the presence of an inducer molecule or the absence of an inhibitor molecule, thereby providing a method to turn off expression of the gene when it is not desired. In yet another embodiment, the promoters are cell-type-specific promoters or tissue-specific (e.g., heart-tissue-specific) promoters. Genes under the control of cell-type-specific promoters will be expressed only in certain cell types, preferably only in cardiomyocytes.

In another embodiment of the present invention, the PDE-associated agent is a modulator (e.g., inhibitor, activator, antagonist, or agonist) of PDE expression/activity, including a modulator of a member of the PDE signal-transduction pathway/system. The modulator of the present invention may be a protein, polypeptide, peptide, nucleic acid (including DNA or RNA), antibody, Fab fragment, F(ab′)2 fragment, molecule, compound, antibiotic, or drug, including an agent reactive with PDE, and an agent that induces or upregulates PDE expression or activity.

Modulators of PDE or a member of the PDE signal-transduction pathway/system may be identified using a simple screening assay. For example, to screen for candidate modulators of PDE, cells may be plated onto microtiter plates, then contacted with a library of drugs. Any resulting increase in, or upregulation of, PDE expression then may be detected directly or indirectly using a luminescence reporter, nucleic acid hybridization, and/or immunological techniques known in the art, including an ELISA. Additional modulators of PDE expression may be identified using screening procedures well known in the art or disclosed herein. It is within the confines of the present invention that the modulator of PDE expression may be linked to another agent, or administered in combination with another agent, such as a drug or a ribozyme, in order to increase the effectiveness of treatments using PDE-associated agents and/or increase the efficacy of targeting. Additional PDE-associated agents may be identified using screening procedures well known in the art, and methods described herein.

As discussed above, the present invention contemplates the use of proteins and protein analogues generated by synthesis of polypeptides in vitro, e.g., by chemical means or in vitro translation of mRNA. For example, PDE may be synthesized by methods commonly known to one skilled in the art (Modem Techniques of Peptide and Amino Acid Analysis (New York: John Wiley & Sons, 1981); Bodansky, M., Principles of Peptide Synthesis (New York: Springer-Verlag New York, Inc., 1984)). Examples of methods that may be employed in the synthesis of the amino acid sequences, and analogues of these sequences, include, but are not limited to, solid-phase peptide synthesis, solution-method peptide synthesis, and synthesis using any of the commercially-available peptide synthesizers. The amino acid sequences of the present invention may contain coupling agents and protecting groups, which are used in the synthesis of protein sequences, and which are well known to one of skill in the art.

In accordance with the method of the present invention, PDE in cells may be modulated, and cells may be contacted with a PDE-associated agent (e.g., by introducing a PDE-associated agent directly into the cells) including stem cells containing a PDE-associated agent either in vitro, or in vivo in a subject. Where cells are contacted with a PDE-associated agent in vitro, the agent may be added directly to the cell-culture medium. Alternatively, a PDE-associated agent may be contacted with cells in vivo in a subject, by introducing the agent into the subject (e.g., by introducing the agent directly into cells of the subject) and/or administering the agent to the subject. The subject may be any animal, including amphibians, birds, fish, mammals, and marsupials, but is preferably a mammal (e.g., a human; a domestic animal, such as a cat, dog, monkey, mouse, and rat; or a commercial animal, such as a cow or pig). In a preferred embodiment, the subject is a human.

The PDE-associated agent of the present invention may be contacted with a cell or RyR complex, either in vitro, or in vivo (including in situ) in a subject, by known techniques used for the introduction and administration of proteins, nucleic acids, and other drugs. Examples of methods for contacting the cells with (i.e., treating the cells with) a PDE-associated agent include, without limitation, absorption, electroporation, immersion, injection (including microinjection), introduction, liposome delivery, stem cell fusion (including embryonic stem cell fusion), transduction, transfection, transfusion, vectors, and other protein-delivery and nucleic-acid-delivery vehicles and methods.

When the cells are localized to a particular portion of a subject, it may be desirable to introduce the agent directly to the cells, by injection or by some other means (e.g., by introducing the agent into the blood or another body fluid). Preferably, where heart tissue cells are contacted with a PDE-associated agent in vivo in a subject, contacting is accomplished via a catheter inserted directly into the subject's heart tissue. A catheter would be useful in achieving targeted delivery of the agent to heart tissue cells. Targeted delivery is especially appropriate for cardiomyocytes, which are joined by intercalated disks. These disks should allow passage of the agent from one cardiomyocyte to adjoining cardiomyocytes, thereby aiding in the distribution of the agent throughout the heart tissue.

Where a PDE-associated agent is a protein, it may be introduced into a cell or RyR complex directly, in accordance with conventional techniques and methods disclosed herein. Additionally, a protein agent may be introduced into a cell or RyR complex indirectly, by introducing into the cells a nucleic acid encoding the agent, in a manner permitting expression of the protein agent. The PDE-associated agent may be introduced into cells, in vitro or in vivo, using conventional procedures known in the art, including, without limitation, electroporation, DEAF dextran transfection, calcium phosphate transfection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of such viruses as retrovirus, HSV, adenovirus, adeno-associated virus, Semilild Forest virus, cytomegalovirus, lentivirus, and vaccinia virus.

The amount of nucleic acid to be used in the method of the present invention is an amount sufficient to express an amount of protein effective to promote PDE level. These amounts may be readily determined by the skilled artisan. It is also within the confines of the present invention to use an ex vivo approach, wherein a nucleic acid encoding a protein agent is introduced into suitable a cell or RyR complex in vitro, using conventional procedures, to achieve expression of the protein agent in the cells. Cells expressing protein agent are then introduced into a subject to promote PDE activity in vivo.

In accordance with the method of the present invention, a PDE-associated agent, including stem cells containing the agent, may be administered to a human or animal subject by known procedures, including, without limitation, oral administration, parenteral administration, transdermal administration, and by way of a catheter. For example, the agent may be administered parenterally, by intracranial, intraspinal, intrathecal, or subcutaneous injection. The agent of the present invention also may be administered to a subject in accordance with any of the above-described methods for effecting in vivo contact between a cell or RyR complex and PDE-associated agents.

For oral administration, a formulation comprising a PDE-associated agent may be presented as capsules, tablets, powders, granules, or as a suspension. The formulation may have conventional additives, such as lactose, mannitol, cornstarch, or potato starch. The formulation also may be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration (i.e., administration by injection through a route other than the alimentary canal) or administration through a catheter, a PDE-associated agent may be combined with a sterile aqueous solution that is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampoules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual, or by way of a catheter.

For transdermal administration, an agent may be combined with skin penetration enhancers, such as propylene glycol, polyethylene glycol, isopropanol, ethanol, oleic acid, N-methylpyrrolidone, and the like, which increase the permeability of the skin to the agent, and permit the agent to penetrate through the skin and into the bloodstream. The agent/enhancer composition also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch.

The method of the present invention may also be used either to treat a RyR receptor associated disorder or disease in vivo in a subject, or to prevent a RyR receptor associated disorder or disease in vivo in a subject. As the inventors have demonstrated, augmented PDE in a cell or RyR complex has the ability protect the heart against heart failure and catecholaminergic arrhythmias by regulating local PKA activity and channel activation at RyR2-Ser²⁸⁰⁹, and preventing excess accumulation of cAMP and uncontrolled PKA activation.

The present invention also provides a therapeutic composition comprising a PDE-associated agent and, optionally, a pharmaceutically-acceptable carrier. As described above, the PDE-associated agent may include a PDE protein or nucleic acid, a PDE-associated protein, a PDE-associated nucleic acid, a member of the PDE signal-transduction pathway (including upstream and downstream effectors and activators, in protein or nucleic acid form), and a modulator (e.g., inhibitor, activator, antagonist, or agonist) of a member of the PDE signal-transduction pathway/system (i.e., a modulator which affects the expression and/or activity of PDE or a member of the PDE signal transduction pathway).

In accordance with the therapeutic composition of the present invention, the pharmaceutically-acceptable carrier must be “acceptable” in the sense of being compatible with the other ingredients of the composition, and not deleterious to the recipient thereof. The pharmaceutically-acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include, without limitation, carboxymethyl cellulose, crystalline cellulose, glycerin, gum arabic, lactose, magnesium stearate, methyl cellulose, powders, saline, sodium alginate, sucrose, starch, talc, and water, among others.

Formulations of the therapeutic composition of the present invention may be prepared by methods well-known in the pharmaceutical arts. For example, a PDE-associated agent may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The PDE-associated agent is provided in an amount that is effective to hydrolyze cAMP in a cell or RyR complex in a subject to whom the therapeutic composition is administered. This amount may be readily determined by the skilled artisan.

In one embodiment of the present invention, the PDE-associated agent is a protein that is expressed in a target heart tissue cell using an expression construct. Expression of the protein may be controlled by methods known in the art, including the use of attenuators, downregulators, inhibitors, and other molecules known to inhibit protein expression. By way of example, where the therapeutic composition of the present invention is administered to a subject, such that the composition expresses a PDE-associated protein in the subject, this expression may be shut off in vivo by subsequently administering to the subject an attenuator, downregulator, inhibitor, or other molecule that will inhibit expression of the exogenous molecule. Control of expression of the PDE-associated protein is also advantageous, in that it allows one to turn off the expression of the protein when desired, thereby minimizing any harmful side-effects in a subject to whom the composition is administered. Continuous expression of such a protein, beyond an appropriate time limit, may harm the subject. For example, a significant interference with a PDE signal transduction pathway may cause neoplasia or apoptosis.

The therapeutic composition of the present invention may further comprise a vehicle for assisting in the delivery of the composition that target specific cells, including but not limited to heart tissue cells or skeletal muscle cells. A variety of biological delivery systems (e.g., antibodies, bacteria, liposomes, and viral vectors) currently exist for delivering drugs, genes, immunostimulators, pro-drug converting enzymes, radiochemicals, and other therapeutic agents to the vicinity of target cells (see e.g., Ng et al., An anti-transferrin receptor-avidin fusion protein exhibits both strong proapoptotic activity and the ability to deliver various molecules into cancer cells, Proc. Natl. Acad. Sci. USA, 99:10706-11, 2002; Mastrobattista et al., Functional characterization of an endosome-disruptive peptide and its application in cytosolic delivery of immunoliposome-entrapped proteins, J. Biol. Chem., 277:27135-43, 2002; Fefer, “Special delivery” to cancer cells, Blood, 99:1503-04, 2002; Kwong et al., The suppression of colon cancer cell growth in nude mice by targeting β-catenin/TCF pathway, Oncogene, 21:8340-46, 2002; Huser et al., Incorporation of decay-accelerating factor into the baculovirus envelope generates complement-resistant gene transfer vectors, Nat. Biotechnol., 19:451-55, 2001; Lu et al., Polymerizable Fab′ antibody fragments for targeting of anticancer drugs, Nat. Biotechnol., 17:1101-04, 1999; and Chu et al., Toward highly efficient cell-type specific gene transfer with retroviral vectors displaying single-chain antibodies, J. Virol. 71:720-25, 1997). For example, U.S. Pat. No. 6,491,905 provides a prokaryotic cell stably carrying a vector that includes a DNA sequence encoding a purine nucleotide phosphorylase or hydrolase, and the use of such a cell, together with a purine pro-drug, to treat tumors.

In one embodiment of the present invention, the vehicle is a liposome. Liposomal vesicles may be prepared by various methods known in the art, and liposome compositions may be prepared using any one of a variety of conventional techniques for liposome preparation known to those skilled in the art. Examples of such methods and techniques include, without limitation, chelate dialysis, extrusion (with or without freeze-thaw), French press, homogenization, microemulsification, reverse phase evaporation, simple freeze-thaw, solvent dialysis, solvent infusion, solvent vaporization, sonication, and spontaneous formation. Preparation of the liposomes may be carried out in a solution, such as an aqueous saline solution, aqueous phosphate buffer solution, or sterile water. Liposome compositions also may be prepared by various processes involving shaking or vortexing.

The therapeutic composition of the present invention may be incorporated into the layers of a liposome, or enclosed within the interior of the liposome. The liposome containing the composition then may be fused cell, in accordance with known methods of fusion of liposomes to cell membranes, such that the composition protein is delivered into the membrane of the cell or into the interior of the cell, as the case may be.

The present invention also provides a kit for use in delivering a PDE-associated agent to a cells, cell subcompartment or RyR complex in a subject. The kit comprises a therapeutic composition and a catheter. As described above, the therapeutic composition may comprise a PDE-associated agent; optionally, a pharmaceutically-acceptable carrier; and, optionally, a liposome, viral vector, or other vehicle.

The present invention is described in the following Examples, which are set forth to aid in the understanding of the invention, and should not be construed to limit in any way the scope of the invention as defined in the claims which follow thereafter.

### EXAMPLES

### Example 1

### PDE4D−/− Mice an RYR2-S2808A Knock in Mice

Mouse genomic λ-phage clones for segments of the murine ortholog of the human RyR2 gene were isolated from a 129/SvEvTacfBR genomic library (Stratagene, La Jolla, Calif.). A 5.4 kb Eco RI fragment containing exons 53 to 55 and the flanking intronic regions was isolated using a 250 bp ³²P-labeled cDNA probe containing serine (S) 2808. The isolated 5.4 kb fragment was subcloned into the Eco RI site of pBluescriptSK, and S2808 was mutated to alanine (A) using a Chameleon Mutagenesis Kit (Stratagene, La Jolla Calif.). In addition to the S2808A mutation, an extra FSP I restriction site was added to exon 55. The 5.4 kb Eco RI fragment was then excised and cloned into the Eco RI site of pACN vector. The pACN plasmid was a backbone vector containing a cassette (ACN) with genes for neomycin resistance, Cre recombinase and a testes-specific promoter (tACE), flanked by loxP sites. The promoter tACE initiates expression of Cre recombinase only during spermatogenesis, resulting in excision of the ACN cassette. The 3′ targeting arm, consisting of the 2463 bps upstream of the Eco RI site, was obtained by PCR of genomic mouse DNA. After adding Sal I sites to both ends of this 2.4 kb fragment, it was cloned into the Sal I site of the pACN vector containing the mutated 5.4 kb segment. The Kpn I linearized targeting vector was electroporated into MM13 mouse embryonic stem (ES) cells using established protocols. Gene-targeted ES cells were screened by Southern analysis using both 5′ and 3′ external probes to confirm homologous recombination, and injected into C57BI6 blastocysts. Founder mice were backcrossed to C57B16 mice. Germline offspring were identified by brown coat color and further verified by Southern blot analysis. Heterozygous males and females were intercrossed to obtain homozygous offspring.

PDE4D^{-/-} mice were generated and genotyped as described (Jin et al., 1999). RyR2-S2808A knockin mice, generated using homologous recombination as described immediately above, exhibited normal cardiac structure and function, and no PKA phosphorylation of RyR2 was detected using a kinasing reaction with [γ-³²P]ATP or with a phosphoepitope-specific antibody that detects PKA-phosphorylated RyR2.

### Example 2

### Transthoracic Echocardiography and In Vivo Hemodynamic Analyses on Mice

Transthoracic 2D echocardiography and in vivo hemodynamic analyses on mice were performed as previously described. All animal studies were performed according to protocols approved by the Institutional Animal Care and Use Committee of Columbia University and according to NIH guidelines. For transthoracic 2-D echocardiography, mice were anesthetized with 1.0-1.5% isoflurane in Oz and placed on a 37° C. heating pad. Hearts were visualized parasternally along the short axis to obtain 2-D images and M-mode tracings of the anterior wall, left ventricular cavity, and posterior wall. Left ventricular dimensions and function were measured in triplicate from different cardiac cycles for the number of animals indicated.

Hemodynamic measurements were performed on PDE4D^{/31 /-}, PDE4D^{+/-} and age-and litter-matched wild-type (WT) mice (3 to 15 months) anesthetized with 1.5% isoflurane using a 1.4 F micromanometer-conductance catheter (SPR-839, Millar Instruments) via the right carotid artery. Pressure-volume analysis was performed using hemodynamic analysis software (EMKA Technologies, VA) as described (van Rooij et al., 2004).

### Example 3

### Myocardial Infarct Model

PDE4D^{-/-} and age-and-litter-matched wild-type mice (4 to 5 months old) were anesthetized with 1.5% isoflurane and ventilated with a small-rodent respirator (Harvard Apparatus). A left thoracotomy was performed, and the left anterior descending artery (LAD) was ligated proximally with an 8-0 suture as described (Wehrens et al., 2005).

### Example 4

### Exercise Testing and Mouse ECG Recording

Ambulatory ECG recordings were performed using implantable radiotelemetry transmitters (DSI) as described (Wehrens et al., 2004). For the pharmacological experiments performed in wt and RyR2-S2808A knockin mice, animals were pretreated for 30 min by intraperitoneal injection with the PDE4 inhibitor rolipram (0.3 mg/kg) or placebo (DMSO 0.5% as carrier) followed by the exercise protocol described above.

### Example 5

### β-Adrenergic Receptor Measurement

β-adrenergic-receptor levels were assessed as previously described (Reiken et al., 2003b). Aliquots of cardiac membrane preparations from 5 wild-type (WT) and 5 PDE4D^{-/-} knockout mice were incubated for 2 hours in 0.5 mM Tris-HCl buffer, pH 7.4, containing increasing concentrations of [¹²⁵I]-(-)-cyanopindolol ([¹²⁵I]-CYP) before the reactions were filtered using GF/C microfiber filters from Whatman. The filters were washed three times with 3 ml of binding buffer, dried, and bound radioligand was measured in a γ-radiation counter. [¹²⁵I]-CYP binding was determined in the presence and absence of 1 μM alprenolol to distinguish between specific and nonspecific (residual binding in the presence of 1 μM alprenolol) binding.

### Example 6

### Immunoprecipitation and Immunoblot Analysis

RyR2 channels were immunoprecipitated and immunoblotted as previously described (Marx et al., 2000). RyR2 channels were immunoprecipitated from 100 μg of human or murine cardiac homogenates using anti-RyR antibody (Jayaraman et al., 1992) in 0.5 ml of RIP A buffer (50 mM Tris-HCl buffer), pH 7.4, 0.9% NaCl, 5.0 mM NaF, 1.0 mM Na₃ V0₄, 0.25% Triton-XI00, and protease inhibitor mix (Roche) overnight at 4° C. The use of human tissues was approved by the Institutional Review Board of Columbia-Presbyterian Medical Center. Normal and failing human heart tissues were obtained as previously described from patients undergoing cardiac transplant (Marx et al, 2000). Immunoprecipitates were separated by SDS-PAGE and the proteins were transferred onto nitrocellulose membranes overnight (Semi-Dry transfer blot, Bio-Rad, USA). Immunoblots were developed with an enhanced chemiluminescence system using primary antibodies against RyR (5029; 1:3,000) (Jayaraman et al., 1992), PDE splice-variant 4D3 (1:1,000) (Reiken et al, 2003b) calstabin2 (1:1,000) (Wehrens et al., 2003), PKA catalytic subunit (1:1,000), PPI (1:1,000) and PP2A (1:1,000) (Transduction Labs, Lexington, Ky.) (Marx et al., 2000). PKA phosphorylation of RyR2 was quantified using phospho-epitope specific RyR2-pSer²⁸⁰⁸ antibody (1:5,000) (Reiken et al., 2003c). Results were confirmed using a PKA back-phosphorylation assay as previously described (Reiken et al., 2003c). CaMKII phosphorylation of RyR2 was quantified using phospho-epitope specific antibody RyR2-pSer²⁸¹⁴ (1:5,000) as described (Wehrens et al., 2004). Band densities were quantified using Quantity One software (Biorad, Hercules, Calif.) (Reiken et al., 2003a). Data presented represent ≧4 individual experiments.

### Example 7

### Phosphodiesterase Activity Assay

Phosphodiesterase (PDE) activity was measured using selective binding of 5′-AMP to yttrium silicate beads with embedded scintillant. Immunoprecipitated RyR2 complexes were incubated with 50 nM ³H-cyclic nucleotide (Amersham, 5-60 Ci/mM) in 50 mM Tris-HCl (pH 7.5), 8.3 mM MgCl₂, 1.7 mM EGTA, BSA 0.01% at 30° C. for 30 min. The reaction was terminated by adding one-third of 5 mg/ml yttrium silicate beads in 18 mM Zn acetate/Zn sulfate solution (3:1). After 30 min, hydrolysis was quantified by a scintillation counter (Wallac 1409, PerkinElmer).

### Example 8

### FRET-PKA Assay

Local intracellular cAMP concentrations were determined in murine cardiomyocytes using fluorescence resonance energy transfer (FRET) between the cyan (CFP) and yellow (YFP) variants of green fluorescent protein as described (Zaccolo and Pozzan, 2002). Primary cultures of adult murine ventricular cardiac myocytes from wild-type and age-and litter-matched PDE4D^{-/-}hearts were isolated using a modified Langendorffperfusion protocol with Ca²⁺ free Tyrode solution followed by collagenase digestion (Type II, Worthington). Cardiomyocytes were infected with recombinant adenoviruses expressing CFP attached to the PKA regulatory subunit (RII-CFP) and YFP attached to the PKA catalytic subunit (C-YFP) as described previously (Warrier et al., 2005). Simultaneous infection of mouse cardiomyocytes occurred at a multiplicity of infection of 50 to 100 for each virus. Cells expressing approximately equal amounts of CFP and YFP as evidenced by fluorescence at 48-72 hrs, were used for intracellular cAMP imaging by FRET. Imaging was performed with an inverted microscope (Olympus IX70) equipped with a 40× water immersion objective (1.3 NA, Olympus) and a CCD camera (Hamamatsu, Orca ER) as described previously (Warrier et al., 2005). Fluorescence images were acquired using 2×2 binning and analyzed using Simple PCI imaging software (Compix Inc.) and changes in cAMP concentrations at the Z-line containing RyR2 complexes were defined as the relative changes in the intensity of CFP and YFP measured at the Z-lines within a region of interest. Isoproterenol bitartrate (Iso; Sigma RBI) was prepared as stock solution and applied by rapid perfusion.

### Example 9

### Back Phosphorylation of PDE4D3

PKA phosphorylation of PDE4D3 was assessed using a kinasing reaction on PDE4D3 immunoprecipitated from 100 82 g of human cardiac homogenates. PDE4D3 was immunoprecipitated from 100 μg of human cardiac homogenates with anti-PDE4D3 antibody (5 μg/ml) in 0.5 ml of RIPA buffer overnight at 4° C. Samples were incubated with Protein A sepharose beads (Amersham Pharmacia Biotech, Piscataway, N.J.) at 4° C. for 1 hour, beads were washed three times with 1× kinase buffer (8 mM MgCl₂, 10 mM EGT A, and 50 mM Tris/piperazine-N,N′-bis(2ethanesulfonic acid), pH 6.8). After resuspending the beads in 10 μl of 1.5× kinase buffer containing PKA catalytic subunit (5 units, Sigma, St. Louis, Mo.), back phosphorylation of the immunoprecipitated PDE4D3 was initiated with 5μ of 100 μM Mg-ATP containing 10% [β-³²P]_A TP (NEN Life Sciences, Boston, Mass.). The reaction was terminated after 10 min at RT with 5 μl of stop solution (4% SDS and 0.25 M DTT). Samples were heated to 95° C., size fractionated on 8% PAGE, and PDE4D3 radioactivity was quantified using a Molecular Dynamics Phosphorimager and ImageQuant software (Ammersham Pharmacia).

### Example 10

### Single Channel Recordings

RyR2 single channels were recorded in planar lipid bilayers as previously described (Marx et al., 2000). Symmetrical solutions used were (in mM) trans-HEPES 250 and Ca(OH)₂ 53 (pH 7.35) and cis-HEPES 250, Tris 125, EGTA 1.0, and CaCl₂ 0.5 (pH 7.35). Free Ca²⁺ concentrations were calculated by CHELATOR software. At the conclusion of each experiment, ryanodine (5 μM) or ruthenium red (20 μM) was applied to confirm RyR2-channel identity.

### Example 11

### PKA Phosphorylation of Cardiac Ryanodine Receptors

PKA phosphorylation of RyR2 was assessed as previously described (Marx et al., 2000). RyR2 was immunoprecipitated from 250 μg of mouse heart homogenate. PKA phosphorylation of RyR2 was initiated with 5 μl of 100 μM Mg-ATP (for autoradiography, the Mg-ATP contained 10% [³²P]-γATP (NEN Life Sciences, Boston, Mass.) in kinase buffer (8 μM MgCl₂, 10 mM EGTA, and 50 mM Tris/piperazine-N,N′-bis(2ethanesulfonic acid), pH 6.8). The reaction was terminated after 8 min at room temperature with 5 μl of stop solution (4% SDS and 0.25 M DTT). Samples were heated to 95 DC and size-fractionated on 6% SDS-PAGE.

### Example 12

### Statistical Analysis

Data are reported as mean ±SEM for in vivo experiments and mean ±SD for biochemical studies. Differences between multiple experimental groups were compared by analysis of variance (ANOVA) followed by Tukey's multiple comparison test. Analysis between two groups was performed by t test (paired or unpaired as appropriate). Serial studies were tested by repeated-measure ANOVA. A value of p<0.05 was considered significant.

### Example 13

PDE4D Gene Inactivation Causes Age-Related Cardiomyopathy

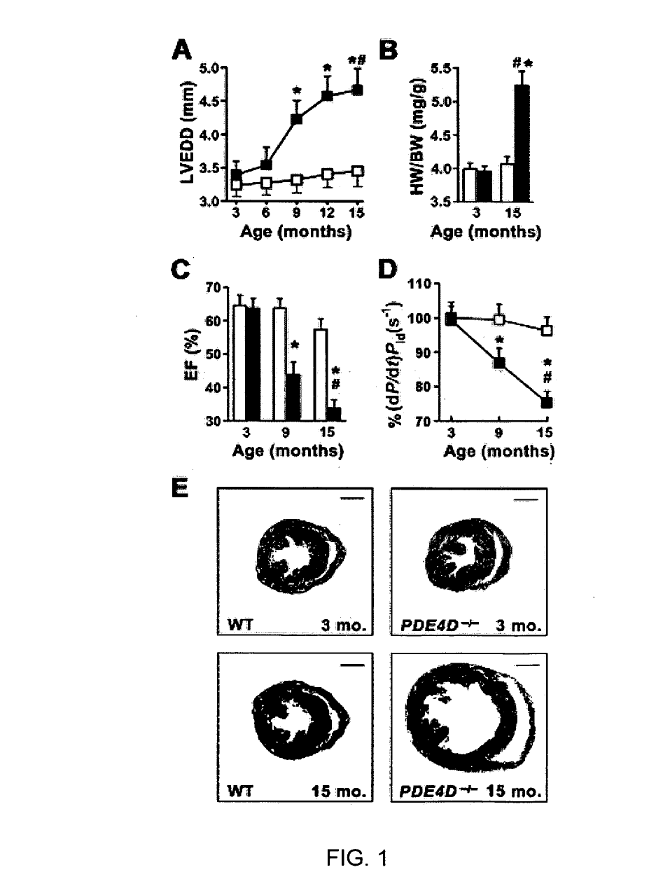

To explore the consequences of chronic PDE4D deficiency on cardiac function, a mouse model of PDE4D gene inactivation (Jin et al., 1999) was used. Echocardiography of PDE4D^{-/-} mice showed a progressive, age-dependent increase in left ventricular end-diastolic diameter (LVEDD), a hallmark of cardiac dysfunction (FIG. 1A; n=12 each for wild-type [wt] and PDE4D^{-/-}). PDE4D^{-/-} mice exhibited increased heart-weight-to-body-weight (HW/BW) ratios compared to wt controls (FIG. 1B). PDE4D^{-/-} mice had reduced ejection fractions (EF) and cardiac contractility (dP/dt)/P_{id}, documented by cardiac catheterization (FIGS. 1C and 1D). Histologic examination of PDE4D^{-/-} hearts confirmed that the 15-month-old hearts were dilated, with no other structural abnormalities (FIG. 1E). These data show that PDE4D deficiency is associated with progressive cardiac dysfunction consistent with a dilated cardiomyopathy similar to that seen in patients with chronic heart failure.

### Example 14

### Global cAMP Signaling is Normal in PDE4D-Deficient Mice

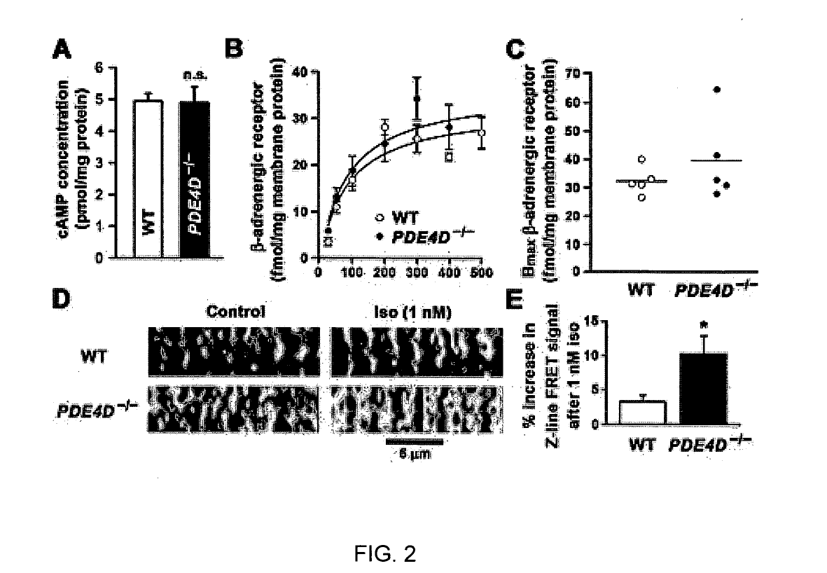

It is well established that chronic hyperadrenergic signaling is associated with heart failure. Therefore, the inventors sought to determine whether the mechanism underlying the observed cardiac phenotype in PDE4D^{-/-} mice was increased global cAMP signaling. However, there were no significant differences in global cAMP levels and β-adrenergic receptors in hearts from PDE4D^{-/-} mice (FIGS. 2A-2C). Total cAMP-hydrolyzing activity of PDE in the heart was only slightly decreased in PDE4D^{-/-} mice (data not shown), consistent with PDE4D activity representing only a fraction of total cytosolic PDE cAMP-hydrolyzing activity in the heart (Mongillo et al., 2004 and Richter et al., 2005). However, rolipram-sensitive PDE4 activity in PDE4D^{-/-} heart was reduced by ˜50% (data not shown). Whereas global cAMP signaling was not perturbed in PDE4D-deficient mice, there was a significant increase in localized cAMP levels at the cardiomyocyte Z line (corresponding to the location of the RyR2 channel) in cardiomyocytes isolated from PDE4D^{-/-} mice following a low dose (1 nM) of isoproterenol (FIGS. 2D and 2E). Thus, the abnormalities observed in cardiac function of PDE4D^{-/-} mice must be explained by defects in localized cAMP-dependent signaling.

### Example 15

### PKA Phosphorylation of RYP2 in PDE4D^{-/-} Mice

While many proteins in the heart are regulated by PKA phosphorylation and therefore can be affected by altered PDE4D gene expression, only a limited number of these PKA substrates are known to be dysregulated by PKA during heart failure. For example, it has been shown that PKA phosphorylation of phospholamban, a regulator of the SR Ca²⁺ uptake pump (SERCA2a), is decreased in heart failure. In contrast, of the other proteins known to be involved in regulating cardiac contractility, the SR Ca²⁺-release channel, RyR2, has been shown to be PKA hyperphosphorylated in heart failure (Antos et al., 2001, Marx et al., 2000, Reiken et al., 2003a, Reiken et al., 2003b, Yano et al., 2000 and Yano et al., 2003), although this finding has been challenged by others (Jiang et al., 2002).

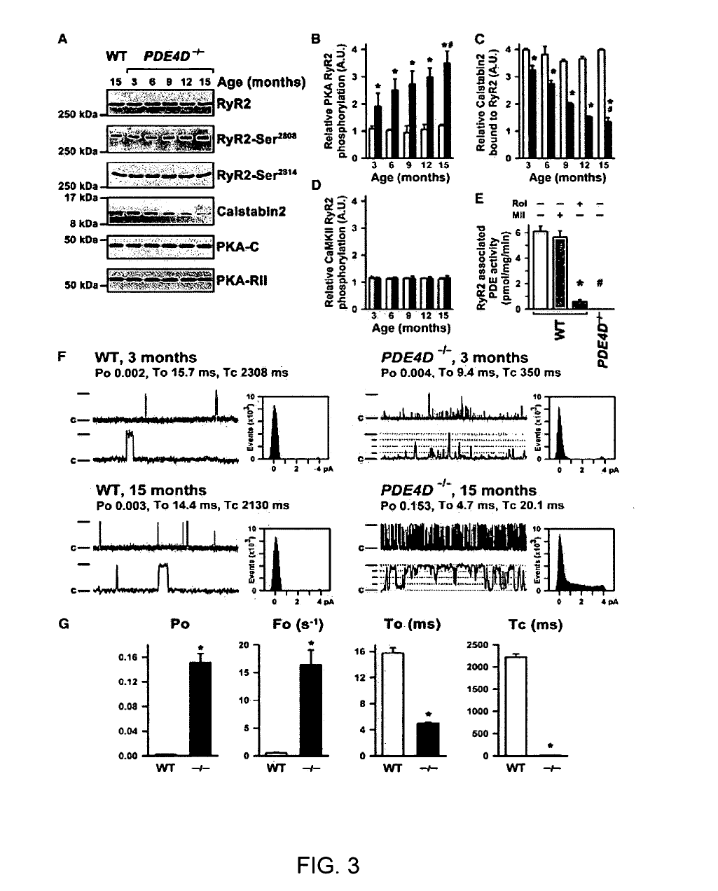

Given that RyR2 PKA hyperphosphorylation has been linked to cardiac dysfunction in humans and animal models and that cAMP concentrations are increased in the compartment of RyR2 Ca²⁺ release (FIGS. 2D and 2E), the inventors sought to determine whether RyR2 PKA hyperphosphorylation might play a role in the observed cardiac phenotype in PDE4D^{-/-} mice. Indeed, there was a progressive, age-dependent increase in PKA phosphorylation of RyR2 on Ser2808 (detected using a phosphoepitope-specific antibody) in PDE4D^{-/-} mouse hearts (FIGS. 3A and 3B). PKA hyperphosphorylation of RyR2 in PDE4D^{-/-} mouse hearts was associated with depletion of the RyR2-stabilizing protein calstabin2 (FKBP12.6) that prevents Ca²⁺ leak from the SR into the cytosol through RyR2 during diastole in the heart (FIG. 3C) (Marx et al., 2000 and Wehrens et al., 2003). Increased PKA phosphorylation of RyR2 was not caused by increased protein levels of PKA catalytic and regulatory subunits in the RyR2 complex (FIG. 3A). Moreover, no changes in PP1 or PP2A phosphatase levels in the RyR2-complex levels were detected (data not shown). Phosphorylation of RyR2 by another kinase that phosphorylates the channel, Ca²⁺/calmodulin-dependent protein kinase A (CaMKII) (detected using a phosphoepitope-specific antibody), was not altered in PDE4D^{-/-} mouse hearts (FIGS. 3A and 3D).

In wt mice, PDE activity was associated with immunoprecipitated RyR2 channels (FIG. 3E). RyR2-associated PDE activity was almost completely inhibited by the PDE4 antagonist rolipram but not by the PDE3 inhibitor milrinone. Moreover, PDE activity specifically associated with RyR2 channels was reduced to zero in the PDE4D^{-/-} mice (FIG. 3E). Taken together, these data suggested that one consequence of PDE4D deficiency is PKA hyperphosphorylation of RyR2, which has previously been associated with heart failure (Marx et al., 2000). Moreover, it appeared that the PDE activity associated with the RyR2 complex was encoded by the PDE4D gene.

### Example 16

### Abnormal RYR2 Channels in PDE4D-Deficient Hearts

To ascertain the functional consequences of PKA hyperphosphorylation of RyR2 in hearts from PDE4D-deficient mice, the inventors examined the single-channel properties of RyR2 in planar lipid bilayers. Compared to channels from wt mice, RyR2 from PDE4D^{-/-} mice exhibited significantly increased open probability (Po) and frequency of openings (Fo) and decreased mean open and closed times when channels were examined under conditions that mimic diastole in the heart (cytosolic (cis) [Ca²⁺] 150 nM) (FIGS. 3F and 3G). Thus, PKA hyperphosphorylation of cardiac RyR2 in PDE4D-deficient mice was associated with the same defects in RyR2-channel function (“leaky” channels) previously linked to human heart failure (Marx et al., 2000) and genetically determined exercise-induced sudden cardiac death (Wehrens et al., 2003).

### Example 17

### PDE4D3 is a of the RYR2 Macromolecular Complex

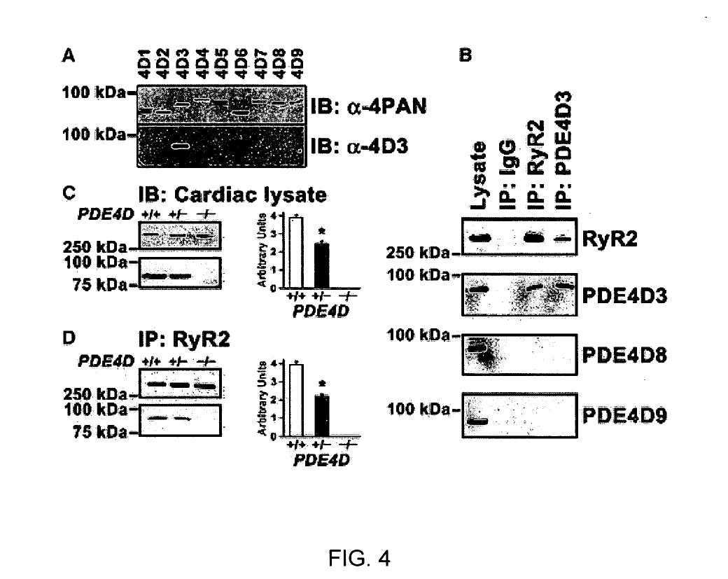

The finding that the PDE activity that coimmunoprecipitates with RyR2 was abrogated in channels from PDE4D^{-/-} mouse hearts (FIG. 3E) raised the possibility that a protein encoded by the PDE4D gene is an integral component of the RyR2 macromolecular signaling complex. Four genes constitute the type 4 phosphodiesterase family (PDE4A, PDE4B, PDE4C, and PDE4D), and all are expressed as multiple splice variants (Conti et al., 2003 and Houslay and Adams, 2003). With the recent discovery of additional PDE4D variants (Gretarsdottir et al., 2003 and Wang et al., 2003), a total of nine PDE4D splice variants (PDE4D1-9) are known (Richter et al., 2005). The inventors generated an isoform-specific antibody against the unique N-terminal epitope of PDE4D3 (FIG. 4A). PDE4D splice variants were identified by RT-PCR in heart (data not shown), and PDE4D3, PDE4D8, and PDE4D9 expression in the heart was demonstrated using isoform-specific antibodies (FIG. 4B), confirming that these are the major PDE4D isoforms expressed in heart muscle (Richter et al., 2005).

To examine the possibility that a specific PDE4D isoform is part of the cardiac RyR2 channel complex, immunoprecipitations were performed using human heart extracts. RyR2 channels were immunoprecipitated with anti-RyR2 antibody and assayed for coimmunoprecipitation of PDE4D3, PDE4D8, or PDE4D9 with RyR2 by immunoblotting. Using isoform-specific PDE4D antibodies, only PDE4D3 was detected in the RyR2 complex. In addition, anti-PDE4D3 antibody was used to coimmunoprecipitate RyR2 (FIG. 4B). The interaction between PDE4D3 and RyR2 was specific because PDE4D3 was excluded from immunoprecipitates using control IgG (FIG. 4B). Moreover, the inventors used the PDE4D3-specific antibody to show that total PDE4D3 protein was decreased ˜37% in haploinsufficient PDE4D^{+/-} mouse hearts and by 100% in homozygous PDE4D^{-/-} hearts (FIG. 4C). Finally, PDE4D3 in the RyR2 macromolecular signaling complex was also decreased by ˜44% in PDE4D^{+/-} mouse hearts and by 100% in PDE4D^{-/-} heart (FIG. 4D). Taken together, these data show that PDE4D3 is an integral component of the RyR2 macromolecular complex in the heart and that PDE4D3 is the only PDE isoform in the RyR2 complex.

### Example 18

### PDE4D3 is Decreased in the RYR2 Complex in Failing Human Hearts

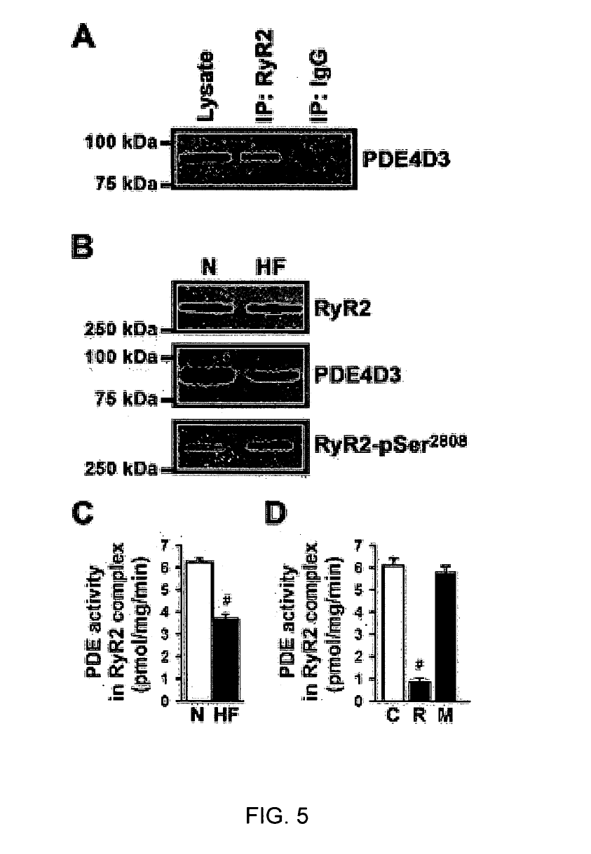

PDE4D3 was also associated with RyR2 from human hearts (FIG. 5A). In human heart failure (HF), PDE4D3 levels in the RyR2 complex were decreased by 43% (normal, n=6 versus HF, n=9; p<0.001), and the RyR2 channels in the human HF samples were PKA hyperphosphorylated (FIG. 5B). The inventors have previously shown that PKA hyperphosphorylation of RyR2 depletes calstabin2 from the RyR2 complex and significantly increases channel activity, consistent with a diastolic SR Ca²⁺ leak in human heart failure (Marx et al., 2000 and Reiken et al., 2003a). The cAMP-hydrolyzing activity of RyR2 bound PDE4D3 was decreased by 42% in human HF samples (n=6, each experiment was performed in triplicate; p<0.001), providing a possible explanation for chronic RyR2-Ser2808 PKA hyperphosphorylation observed in failing human hearts (FIG. 5C). This reduction in PDE4D3 activity in the human HF RyR2 complex was comparable to that observed in the RyR2 complexes in PDE4D^{+/-} mice (FIG. 4D). To explore the basis for the observed reduction in PDE4D3 amount and activity in the HF RyR2 complex, the inventors examined PKA phosphorylation of PDE4D3, which has been shown to increase its activity (Sette and Conti, 1996) and binding to mAKAP (Carlisle Michel et al., 2004). The inventors observed a ˜40% reduction in PKA phosphorylation of PDE4D3 in the human HF RyR2 complexes compared to nonfailing controls (n=3, p<0.01), providing a possible explanation for the observed decrease in amount and activity of PDE4D3 in the HF RyR2 complexes (data not shown).

PDE4-specific inhibition with rolipram (10 μM) significantly decreased RyR2 bound PDE4D3 activity in normal human heart lysates (n=3, p<0.01), whereas the PDE3-specific inhibitor milrinone (10 μM) had no effect on RyR2-associated PDE activity (FIG. 5D), confirming that the cAMP-hydrolyzing activity in the RyR2 complex is due to PDE4. Thus, PDE4D3 is part of the human RyR2 signaling complex, and reduction of PDE4D3 activity in heart failure may contribute to RyR2 PKA hyperphosphorylation and diastolic SR Ca²⁺ leak observed in failing hearts (Shannon et al., 2003).

### Example 19

### Cardiac Arrhythmias Due to PDE4 Inhibition are Suppressed in Mice Harboring RYR2 that cannot be PKA Phosphorylated

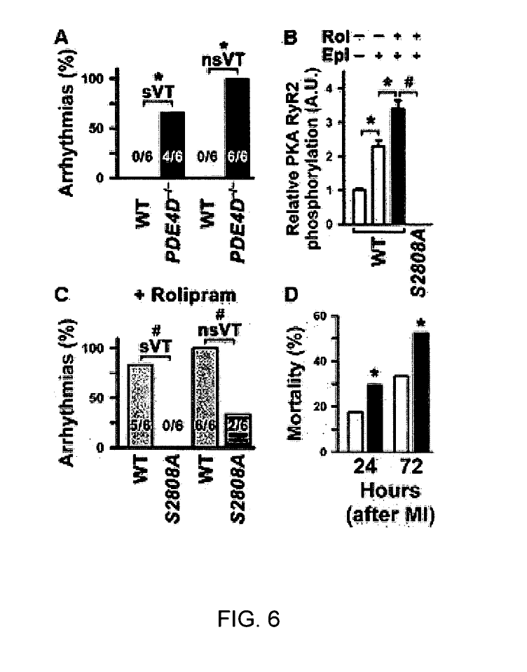

As discussed above, the inventors have previously demonstrated a link between PKA hyperphosphorylation of RyR2, “leaky” RyR2 channels, and exercise-induced sudden cardiac death (Wehrens et al., 2003). Therefore, in the present study, the inventors sought to determine whether PDE4D-deficient mice, which exhibit PKA-hyperphosphorylated RyR2, are more susceptible to exercise-induced cardiac arrhythmias. Resting heart rate in PDE4D^{-/-} mice at 3-4 months of age was similar to wt, consistent with unchanged baseline sympathetic activity (wt 584±22 bpm, PDE4D^{-/-} 603±32 bpm; p=NS). Since PDE inhibitors increase arrhythmogenic sudden cardiac death (Barnes, 2003 and Packer et al., 1991), the inventors tested the susceptibility of PDE4D-/- mice to cardiac arrhythmias during exercise followed by low-dose epinephrine injection (0.1 mg/kg) using a previously established protocol (Wehrens et al., 2003 and Wehrens et al., 2004). Exercise-induced sustained (sVT) and nonsustained ventricular arrhythmias (nsVT) were observed in 66% and 100% of PDE4D^{-/-} mice, respectively, but in none of the wt mice (FIG. 6A; each n=6, p<0.01).

In order to investigate whether a diastolic SR Ca²⁺ leak due to PKA hyperphosphorylation of RyR2 directly contributes to a cardiac phenotype in PDE4D^{-/-} mice, the inventors treated wt mice with the PDE4 inhibitor rolipram. Rolipram (0.3 mg/kg) inhibited cAMP-hydrolyzing PDE activity in the RyR2 complex in wt mice (data not shown) and resulted in significantly increased RyR2 PKA phosphorylation during exercise (FIG. 6B). Following exercise and epinephrine injection (0.1 mg/kg), ventricular arrhythmias or sudden death occurred in 100% of rolipram-treated wt mice (FIG. 6C). Importantly, RyR2-S2808A knockin mice, which express a mutant RyR2 that cannot be PKA phosphorylated (FIG. 6B), were protected against rolipram-induced exercise-triggered arrhythmias (FIG. 6C). These findings indicate that the proarrhythmogenic effects of PDE4 inhibition are specifically due to PKA hyperphosphorylation of RyR2 at Ser2808. Thus, PKA phosphorylation of RyR2 at Ser2808 is necessary in order to generate triggered cardiac arrhythmias associated with PDE4 inhibition. Moreover, mortality due to sudden cardiac death at 24 and 72 hr following myocardial infarction (MI, induced by ligation of the left anterior descending artery) was significantly increased in PDE4D^{+/-} compared to wt mice (FIG. 6D), further suggesting that PDE deficiency in the RyR2-channel complex increases susceptibility to cardiac arrhythmias.

### Example 20

### Exacerbation of Acute Heart Failure Associated with PDE4D3 Deficiency is Attenuated in Mice Harboring RYR2 that cannot be PKA Phosphorylated

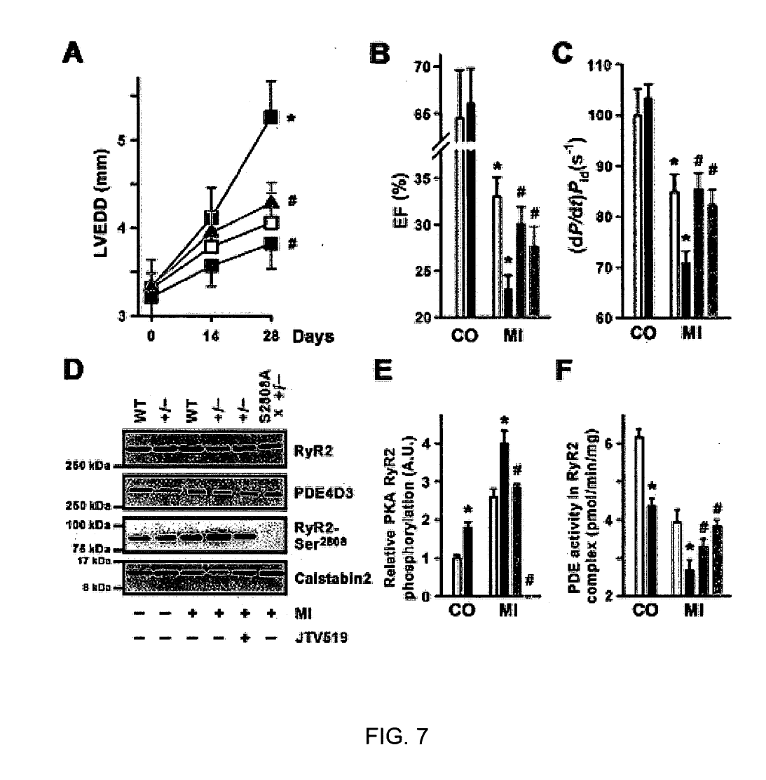

Since PDE4D3 protein levels and cAMP-hydrolyzing activity in the RyR2 complex were reduced by 42% and 43% in human heart failure, respectively, the inventors examined whether a partial reduction of PDE4D in haploinsufficient PDE4D^{+/-} mice affects progression of HF. Heterozygous PDE4D^{+/-} and wt control mice were subjected to proximal left anterior descending (LAD) coronary artery ligation to induce myocardial infarction (MI), which results in progressive heart failure. Similar to human heart failure (FIG. 5B), PDE4D^{+/-} mice had a 44% reduction of PDE4D3 bound to the RyR2 complex as compared to control (FIG. 4D) and developed significantly worse heart failure manifested by a larger increase in cardiac dimensions and more depressed cardiac contractility over a 28 day post-MI period (FIG. 7). Cardiac dimensions (LVEDD) were 30% larger in PDE4D^{+/-} hearts compared to wt 28 days post-MI (FIG. 7A), consistent with more severe cardiomyopathy. Infarct sizes were not significantly different in 4- to 5-month-old wt (35.8%±3.1% LV, n=11) and PDE4D^{+/-} mice (37.2%±3.7% LV, n=14). Cardiac function, measured by echocardiography and cardiac catheterization, was reduced in haploinsufficient PDE4D^{+/-} mice compared with wt mice following MI (FIGS. 7B and 7C). Accelerated HF progression in PDE4D^{+/-} mice was associated with enhanced RyR2 PKA hyperphosphorylation and reduced PDE4D3 protein levels in the RyR2 complex (FIGS. 7D and 7E) and significantly reduced PDE4D3 enzymatic activity in the RyR2 complex (FIG. 7F). In the RyR2 complex, PKA catalytic and regulatory subunits, as well as the levels of the protein phosphatases PP1 and PP2A, were not significantly different between wt and PDE4D^{+/-} hearts (data not shown). Thus, reduction of PDE4D3 activity in the RyR2 complex in haploinsufficient PDE4D^{+/-} mice to levels similar to those observed in RyR2 complexes from failing human hearts results in accelerated progression of heart failure.

Next, the inventors set out to determine whether the detrimental effects of PDE4D deficiency in the heart were dependent on dysfunction of the RyR2-channel complex due to PKA hyperphosphorylation and reduced binding of calstabin2. Since recent studies have demonstrated that the 1,4-benzothiazepine JTV-519 increases the binding of calstabin2 to RyR2 in vivo, the inventors treated PDE4D^{+/-} mice subjected to myocardial infarction with JTV-519. Treatment with JTV-519 (symbols) significantly increased the amount of calstabin2 bound to RyR2 (FIG. 7D) and was associated with improved cardiac function following MI (FIGS. 7B and 7C). The inventors also crossed the PDE4D^{+/-} mice with RyR2-S2808A mice to investigate the specific role of PKA hyperphosphorylation of RyR2 in the development of cardiac dysfunction in PDE4D^{+/-} mice. RyR2-S2808A mice harbor RyR2 that cannot be PKA phosphorylated (FIG. 7D). Prevention of PKA hyperphosphorylation improved cardiac function in PDE4D^{+/-} mice subjected to MI (FIGS. 7A and 7C, green line and bars). Infarct sizes were not significantly different between PDE4D^{+/-} mice, PDE4D^{+/-} mice treated with JTV-519 (38.6%±3.9% LV, n=12), or PDE4D^{+/-} mice crossed with RyR2-S2808A mice (39.8%±4.3% LV, n=11). These data demonstrate that normalizing RyR2 function, either by enhancing calstabin2 binding to RyR2 or by preventing PKA hyperphosphorylation of RyR2, improved cardiac function in PDE4D^{+/-} mice following myocardial infarction. Taken together, these data suggest that the cardiac defects observed in PDE4D-deficient mice are due, at least in part, to defective RyR2 function.

### Example 21

### Summary of Results

The present study shows that phosphodiesterase (PDE4D) deficiency is associated with a severe cardiac phenotype consisting of heart failure and lethal cardiac arrhythmias. The importance of this cardiac phenotype in PDE4D-deficient mice is underscored by the fact that it is similar to that observed in humans with heart failure: decreased cardiac function and increased susceptibility to cardiac arrhythmias. Moreover, PDE inhibition has been associated with increased mortality in patients with heart failure (Packer et al., 1991), arrhythmias, and sudden cardiac death (Bittar and Friedman, 1991 and Suissa et al., 1996), although the mechanism has been unknown. Finally, since PDE4 inhibitors are being tested in clinical trials to treat common chronic diseases including Alzheimer's disease (Gong et al., 2004), asthma, and COPD (Giembycz, 2002), it is important to understand the consequences of long-term inhibition of PDE4 activity in the heart, where PDE4 activity plays a major role in regulating cAMP-dependent signals (Perry et al., 2002, Verde et al., 1999 and Xiang et al., 2005).

Since the PDE4D deficiency caused no detectable alteration in global cAMP levels or β-adrenergic signaling in the heart, the cardiac phenotype in PDE4D^{-/-} mice must be due to abnormalities in localized signaling, i.e., altered microdomains of cAMP, which is supported by the FRET imaging data showing increased cAMP concentrations at the Z lines of PDE4D-deficient cardiomyocytes (where RyR2 is present) after physiologic stimulation of β-adrenergic receptors. Indeed, the concept that localized signaling regulates cAMP in the heart is supported by previous findings showing that PDE4 is a localized regulator of β-adrenergic receptor (β2-AR) signaling in cardiomyocytes (Baillie et al., 2003, Mongillo et al., 2004, Perry et al., 2002 and Xiang et al., 2005). Moreover, it has been shown that PDE4D3 can be targeted to specific compartments including the cardiomyocyte Z line via the targeting protein mAKAP (Carlisle Michel et al., 2004, Dodge et al., 2001, Sette and Conti, 1996 and Yang et al., 1998). These experiments were carefully designed to examine differences in cAMP levels at the Z line in cardiomyocytes from wt versus PDE4D-deficient mice using low-dose β-adrenergic stimulation (1 nM isoproterenol). Others have shown that nonspecific pharmacologic PDE inhibition using maximal β-adrenergic stimulation causes cAMP spillover into different compartments (Zaccolo and Pozzan, 2002).

There are likely many changes in cAMP-dependent signaling in the hearts of PDE4D-deficient mice. For example, receptor-stimulated β-arrestin-mediated recruitment of PDE4 regulates G protein switching by the β2-AR in cardiomyocytes (Baillie et al., 2003). Moreover, PDE4D is an integral component of the β2-AR signaling complex (Xiang et al., 2005). Loss of other PDE4D isoforms (e.g., PDE4D8 and PDE4D9) likely also contributes to localized alterations in cAMP levels in PDE4D^{-/-} cardiomyocytes (Richter et al., 2005). This work shows that PKA phosphorylation of RyR2, which occurs at an early stage, before any structural or functional abnormalities were observed in PDE4D^{-/-} mouse hearts (FIG. 3), is a critical event since crossing the PDE4D-deficient mice with RyR2-S2808A mice inhibits the development of the cardiac phenotype (FIG. 6 and FIG. 7).

Indeed, there are at least two lines of evidence that strongly suggest that the cardiac phenotype in PDE4D-deficient mice is due to defects related to PKA hyperphosphorylation of RyR2 and the resulting abnormal regulation of this channel required for EC coupling in the heart. First, the levels of PDE4D3 in RyR2-channel complexes from failing human hearts are reduced to the same degree as in RyR2 complexes in the hearts of PDE4D^{+/-} mice, which develop accelerated heart failure following myocardial infarction. This suggests that PDE4D deficiency in the RyR2 complex may play a role in PKA hyperphosphorylation of the channel and the associated cardiomyopathy. Second, and more importantly, sustained cardiac arrhythmias associated with pharmacologic PDE4 inhibition and accelerated progression of heart failure following MI were not observed in RyR2-S2808A mice harboring a RyR2 that cannot be PKA phosphorylated (FIG. 6 and FIG. 7). Thus, the present study indicates that the cardiac phenotype in PDE4D-deficient mice is directly related to defective regulation of RyR2 and provides support for the model whereby PKA hyperphosphorylation of RyR2 causes a diastolic SR Ca²⁺ leak that (1) depletes SR Ca²⁺, contributing to decreased cardiac function (Marx et al., 2000), and (2) may provide a trigger for fatal cardiac arrhythmias (Wehrens et al., 2003).

Since the role of PKA hyperphosphorylation of RyR2 in heart failure (Jiang et al., 2002) has been challenged, it is important to establish a mechanism underlying the PKA hyperphosphorylation of RyR2 and to show that this defect can specifically account for the observed cardiac phenotype. The inventors now show that PDE4D3 deficiency in the RyR2 complex contributes to PKA hyperphosphorylation of RyR2 in human and animal heart failure. Furthermore, a mutant RyR2 that cannot be PKA phosphorylated (RyR2-S2808A) protects against the cardiac effects of PDE4D3 deficiency in the RyR2 complex in vivo. Indeed, the finding that PDE4D3 is decreased in RyR2 complexes in human heart failure helps address one of the controversial issues in this field, the mechanism whereby RyR2 become PKA hyperphosphorylated (Marx et al., 2000) despite decreases in global cAMP levels in failing human hearts (Regitz-Zagrosek et al., 1994).

The current findings suggest a novel function of PDE4D3 in the regulation of RyR2, the major intracellular Ca²⁺-release channel in the heart. PDE4D3 activity provides an important negative-feedback mechanism to limit β-AR-dependent PKA phosphorylation of RyR2-Ser2808. Under physiologic conditions, PDE4D3 may regulate local PKA activity and channel activation via phosphorylation of RyR2-Ser2808, thereby preventing excess accumulation of cAMP (Zaccolo and Pozzan, 2002) and uncontrolled PKA-mediated activation of the channel. In human heart failure, loss of negative feedback due to PDE4D3 deficiency in the RyR2 complex likely contributes to RyR2 PKA hyperphosphorylation; calstabin2 depletion; and hyperactive, “leaky” RyR2 channels (Marx et al., 2000 and Pieske et al., 1999). Taken together, these data suggest that PDE4D3 plays a protective role in the heart against heart failure and arrhythmias.

These data further suggest that chronic pharmacologic PDE4 inhibition could contribute to a cardiac phenotype including cardiac dysfunction and arrhythmias, particularly in individuals with underlying cardiac disease. In addition, other signaling systems may be affected by reduced PDE4D activity, e.g., β-arrestin targeting of PDE4D3 activity may be important for P2-AR desensitization (Perry et al., 2002). Importantly, PDE4D3 deficiency and pharmacologic PDE4 inhibition with rolipram was associated with stress-induced cardiac arrhythmias, which did not occur in mice lacking the RyR2 PKA phosphorylation site at Ser2808. These findings suggest that PDE4 inhibitors could increase the risk of cardiac arrhythmias due to “leaky” RyR2 channels as observed in individuals with genetic forms of sudden cardiac death linked to RyR2 mutations (Lehnart et al., 2004 and Wehrens et al., 2003) and in patients with heart failure. The present study shows that phosphodiesterase (PDE4D) deficiency is associated with a severe cardiac phenotype consisting of heart failure and lethal cardiac arrhythmias. The importance of this cardiac phenotype in PDE4D-deficient mice is underscored by the fact that it is similar to that observed in humans with heart failure: decreased cardiac function and increased susceptibility to cardiac arrhythmias. Moreover, PDE inhibition has been associated with increased mortality in patients with heart failure (Packer et al., 1991), arrhythmias, and sudden cardiac death (Bittar and Friedman, 1991 and Suissa et al., 1996), although the mechanism has been unknown. Finally, since PDE4 inhibitors are being tested in clinical trials to treat common chronic diseases including Alzheimer's disease (Gong et al., 2004), asthma, and COPD (Giembycz, 2002), it is important to understand the consequences of long-term inhibition of PDE4 activity in the heart, where PDE4 activity plays a major role in regulating cAMP-dependent signals (Perry et al., 2002, Verde et al., 1999 and Xiang et al., 2005).

Since the PDE4D deficiency caused no detectable alteration in global cAMP levels or P-adrenergic signaling in the heart, the cardiac phenotype in PDE4D^{-/-} mice must be due to abnormalities in localized signaling, i.e., altered microdomains of cAMP, which is supported by the FRET imaging data showing increased cAMP concentrations at the Z lines of PDE4D-deficient cardiomyocytes (where RyR2 is present) after physiologic stimulation of β-adrenergic receptors. Indeed, the concept that localized signaling regulates cAMP in the heart is supported by previous findings showing that PDE4 is a localized regulator of β-adrenergic receptor (p2-AR) signaling in cardiomyocytes (Baillie et al., 2003, Mongillo et al., 2004, Perry et al., 2002 and Xiang et al., 2005). Moreover, it has been shown that PDE4D3 can be targeted to specific compartments including the cardiomyocyte Z line via the targeting protein mAKAP (Carlisle Michel et al., 2004, Dodge et al., 2001, Sette and Conti, 1996 and Yang et al., 1998). The inventors' experiments were carefully designed to examine differences in cAMP levels at the Z line in cardiomyocytes from wt versus PDE4D-deficient mice using low-dose β-adrenergic stimulation (1 nM isoproterenol). Others have shown that nonspecific pharmacologic PDE inhibition using maximal β-adrenergic stimulation causes cAMP spillover into different compartments (Zaccolo and Pozzan, 2002).

There are likely many changes in cAMP-dependent signaling in the hearts of PDE4D-deficient mice. For example, receptor-stimulated β-arrestin-mediated recruitment of PDE4 regulates G protein switching by the p2-AR in cardiomyocytes (Baillie et al., 2003). Moreover, PDE4D is an integral component of the p2-AR signaling complex (Xiang et al., 2005). Loss of other PDE4D isoforms (e.g., PDE4D8 and PDE4D9) likely also contributes to localized alterations in cAMP levels in PDE4D^{-/-} cardiomyocytes (Richter et al., 2005). The inventors' work shows that PKA phosphorylation of RyR2, which occurs at an early stage, before any structural or functional abnormalities were observed in PDE4D^{-/-} mouse hearts (FIG. 3), is a critical event since crossing the PDE4D-deficient mice with RyR2-S2808A mice inhibits the development of the cardiac phenotype (FIG. 6 and FIG. 7).

Indeed, there are at least two lines of evidence that strongly suggest that the cardiac phenotype in PDE4D-deficient mice is due to defects related to PKA hyperphosphorylation of RyR2 and the resulting abnormal regulation of this channel required for EC coupling in the heart. First, the levels of PDE4D3 in RyR2-channel complexes from failing human hearts are reduced to the same degree as in RyR2 complexes in the hearts of PDE4D^{+/-} mice, which develop accelerated heart failure following myocardial infarction. This suggests that PDE4D deficiency in the RyR2 complex may play a role in PKA hyperphosphorylation of the channel and the associated cardiomyopathy. Second, and more importantly, sustained cardiac arrhythmias associated with pharmacologic PDE4 inhibition and accelerated progression of heart failure following MI were not observed in RyR2-S2808A mice harboring a RyR2 that cannot be PKA phosphorylated (FIG. 6 and FIG. 7). Thus, the present study indicates that the cardiac phenotype in PDE4D-deficient mice is directly related to defective regulation of RyR2 and provides support for the model whereby PKA hyperphosphorylation of RyR2 causes a diastolic SR Ca²⁺ leak that (1) depletes SR Ca²⁺, contributing to decreased cardiac function (Marx et al., 2000), and (2) may provide a trigger for fatal cardiac arrhythmias (Wehrens et al., 2003).

Since the role of PKA hyperphosphorylation of RyR2 in heart failure (Jiang et al., 2002) has been challenged, it is important to establish a mechanism underlying the PKA hyperphosphorylation of RyR2 and to show that this defect can specifically account for the observed cardiac phenotype. The inventors now show that PDE4D3 deficiency in the RyR2 complex contributes to PKA hyperphosphorylation of RyR2 in human and animal heart failure. Furthermore, a mutant RyR2 that cannot be PKA phosphorylated (RyR2-S2808A) protects against the cardiac effects of PDE4D3 deficiency in the RyR2 complex in vivo. Indeed, the finding that PDE4D3 is decreased in RyR2 complexes in human heart failure helps address one of the controversial issues in this field, the mechanism whereby RyR2 become PKA hyperphosphorylated (Marx et al., 2000) despite decreases in global cAMP levels in failing human hearts (Regitz-Zagrosek et al., 1994).

The current findings suggest a novel function of PDE4D3 in the regulation of RyR2, the major intracellular Ca²⁺-release channel in the heart. PDE4D3 activity provides an important negative-feedback mechanism to limit β-AR-dependent PKA phosphorylation of RyR2-Ser2808. Under physiologic conditions, PDE4D3 may regulate local PKA activity and channel activation via phosphorylation of RyR2-Ser2808, thereby preventing excess accumulation of cAMP (Zaccolo and Pozzan, 2002) and uncontrolled PKA-mediated activation of the channel. In human heart failure, loss of negative feedback due to PDE4D3 deficiency in the RyR2 complex likely contributes to RyR2 PKA hyperphosphorylation; calstabin2 depletion; and hyperactive, “leaky” RyR2 channels (Marx et al., 2000 and Pieske et al., 1999). Taken together, these data suggest that PDE4D3 plays a protective role in the heart against heart failure and arrhythmias.

These data further suggest that chronic pharmacologic PDE4 inhibition could contribute to a cardiac phenotype including cardiac dysfunction and arrhythmias, particularly in individuals with underlying cardiac disease. In addition, other signaling systems may be affected by reduced PDE4D activity, e.g., β-arrestin targeting of PDE4D3 activity may be important for β2-AR desensitization (Perry et al., 2002). Importantly, PDE4D3 deficiency and pharmacologic PDE4 inhibition with rolipram was associated with stress-induced cardiac arrhythmias, which did not occur in mice lacking the RyR2 PKA phosphorylation site at Ser2808. These findings suggest that PDE4 inhibitors could increase the risk of cardiac arrhythmias due to “leaky” RyR2 channels as observed in individuals with genetic forms of sudden cardiac death linked to RyR2 mutations (Lehnart et al., 2004 and Wehrens et al., 2003) and in patients with heart failure.

While the foregoing invention has been described in some detail for purposes of clarity and understanding, it will be appreciated by one skilled in the art, from a reading of the disclosure, that various changes in form and detail can be made without departing from the true scope of the invention in the appended claims.

### REFERENCES

Antos et al., Dilated cardiomyopathy and sudden death resulting from constitutive activation of protein kinase A, Circ. Res. 89, 997-1004 (2001).

Baille et al., beta-Arrestin-mediated PDE4 cAMP phosphodiesterase recruitment regulates beta-adrenoceptor switching from Gs to Gi. Proc. Natl. Acada. Sci. USA 100, 940-945 (2003).

Barnes, P. J., Theophylline: new perspectives for an old drug. Am. J. Respir. Crit. Care Med. 167, 813-8 (2003).

Bittar et al., The arrhythmogeneicity of theophylline. A multivariate analysis of clinical determinants. Chest 99, 1415-1420 (1991).

Bolger et al., Characterization of five different proteins produced by alternatively spliced mRNAs from the human cAMP-specific phosphodiesterase PDE4D gene. Biochem. J. 328 (Pt 2), 539-48 (1997).

Bristow et al., Beta 1- and beta 2-adrenergic-receptor subpopulations in nonfailing and failing human ventricular myocardium: coupling of both receptor subtypes to muscle contraction and selective beta I-receptor down-regulation in heart failure. Circ. Res. 59, 297-309 (1986).

Carlisle Michel et al., PKA-phosphorylation of PDE4D3 facilitates recruitment of the mAKAP signaling complex. Biochem. J. 381, 587-592 (2004).

Cohn et al., Plasma norepinephrine as a guide to prognosis in patients with chronic congestive heart failure. N. Engl. J. Med. 311, 819-23 (1984).

Conti et al., Cyclic AMP-specific PDE4 phosphodiesterases as critical components of cyclic AMP signaling. J. Biol. Chem. 278, 5493-6 (2003).

Dodge et al., mAKAP assembles a protein kinase A/PDE4 phosphodiesterase cAMP signaling module. EMBO J. 20, 1921-30, (2001).

Feldman et al., Deficient production of cyclic AMP: pharmacologic evidence of an important cause of contractile dysfunction in patients with end-stage heart failure. Circulation 75, 331-9 (1987).

Franzini-Armstrong et al., Shape, size, and distribution of Ca(2+) release units and couplons in skeletal and cardiac muscles. Biophys. J. 77, 1528-39 (1999).

Giembycz, M. A., Development status of second generation PDE4 inhibitors for asthma and COPD: the story so far. Monaldi. Arch. Chest Dis. 57, 48-64 (2002).

Gong et al., Persistent improvement in synaptic and cognitive functions in an Alzheimer mouse model after rolipram treatment. J. Clin. Invest. 114, 1624-1634 (2004).

Gretarsdottir et al., The gene encoding phosphodiesterase 4D confers risk of ischemic stroke. Nat. Genet. 35, 131-8 (2003).

Houslay, et al., PDE4 cAMP phosphodiesterases: modular enzymes that orchestrate signaling cross-talk, desensitization and compartmentalization. Biochem. J. 370, 1-8 (2003).

Jiang et al., Abnormal Ca²⁺ release, but normal ryanodine receptors, in canine and human heart failure. Circ. Res. 91, 1015-1022 (2002).

Jayaraman et al., FK506 binding protein associated with the calcium release channel (ryanodine receptor). J. Biol. Chem. 267, 9474-7 (1992).

Jin et al., Impaired growth and fertility of cAMP-specific phosphodiesterase PDE4D-deficient mice. Proc. Natl. Acad. Sci. USA 96, 11998-2003 (1999).

Lehnart et al., Sudden death in familial polymorphic ventricular tachycardia associated with calcium release channel (ryanodine receptor) leak. Circulation 109, 3208-14 (2004).

Marx et al., Phosphorylation-dependent regulation of ryanodine receptors: a novel role for leucine/isoleucine zippers. J. Cell. Biol. 153, 699-708 (2001).

Marx et al., PKA phosphorylation dissociates FKBP12.6 from the calcium release channel (ryanodine receptor): defective regulation in failing hearts. Cell 101, 365-76 (2000).

Mongillo et al., Fluorescence resonance energy transfer-based analysis of cAMP dynamics in live neonatal rat cardiac myocytes reveals distinct functions of compartmentalized phosphodiesterases. Circ. Res., 95, 67-75 (2004).

Packer et al., Effect of oral milrinone on mortality in severe chronic heart failure. The PROMISE Study Research Group. N. Engl. J. Med. 325, 1468-75 (1991).

Perry et al., Targeting of cyclic AMP degradation to beta 2-adrenergic receptors by beta-arrestins. Science 298, 834-6 (2002).

Pieske et al., Ca²⁺ handling and sarcoplasmic reticulum Ca²⁺ content in isolated failing and nonfailing human myocardium. Circ. Res. 85, 38-46 (1999).

Regitz-Zagrosek et al., Myocardial cyclic AMP and norepinephrine content in human heart failure. Eur. Heart J. 15 (Suppl. D) 7-13. (1994).

Reiken et al., Beta-blockers restore calcium release channel function and improve cardiac muscle performance in human heart failure. Circulation 107, 2459-66 (2003).

Reiken et al., Protein kinase A phosphorylation of the cardiac calcium release channel (ryanodine receptor) in normal and failing hearts. Role of phosphatases and response to isoproterenol. J. Biol. Chem. 278, 444-53 (2003).

Richter et al., Splice variants of the cyclic nucleotide phosphodiesterase PDE4D are differentially expressed and regulated in rat tissue. Biochem. N. 388, 803-811 (2005).

Ruehr et al., Targeting the protein kinase A by muscle A kinase-anchoring protein (mAKAP) regulates phosphorylation and function of the skeletal muscle ryanodine receptor. J. Biol. Chem. 278, 24831-24836. (2003).

Sette et al., Phosphorylation and activation of a cAMP-specific phosphodiesterase by the cAMP-dependent protein kinase. Involvement of serine 54 in the enzyme activation. J. Biol. Chem. 271, 16526-34 (1996).

Sette et al., The ratPDE3/IVd phosphodiesterase gene codes for multiple proteins differentially activated by cAMP-dependent protein kinase. J. Biol. Chem. 269, 18271-4 (1994).

Shannon et al., Elevated sarcoplasmic reticulum Ca²⁺ leak in intact ventricular myocytes from rabbits in heart failure. Circ. Res. 93, 592-4 (2003).

Suissa et al., Bronchodilators and acute cardiac death. Am. J. Respir. Crit. Care Med. 154, 1598-1602 (1996)

Tasken et al., Phosphodiesterase 4D and protein kinase a type II constitute a signaling unit in the centrosomal area. J. Biol. Chem. 276, 21999-2002 (2001).

van Rooij et al,. MCIPI overexpression suppresses left ventricular remodeling and sustains cardiac function after myocardial infarction. Circ. Res. 94, e18-26 (2004).

Verde et al., Characterization of the cyclic nucleotide phosphodiesterase subtypes involved in the regulation of the L-type Ca²⁺ current in rat ventricular myocytes. Br. J. Pharmacol. 127, 65-74 (1999).

Vignola, A. M., PDE4 inhibitors in COPD—a more selective approach to treatment. Respir. Med. 98, 495-503 (2004).

Wang et al., Cloning and characterization of novel PDE4D isoforms PDE4D6 and PDE4D7. Cell. Signal. 15, 883-891 (2003).

Wehrens et al,. FKBPI2.6 deficiency and defective calcium release channel (ryanodine receptor) function linked to exercise-induced sudden cardiac death. Cell 113, 829-40 (2003).

Wehrens et al., Protection from cardiac arrhythmia through ryanodine receptor-stabilizing protein calstabin2. Science 304, 292-6 (2004).

Wehrens et al., Enhancing calstabin binding to ryanodine receptors improves cardiac and skeletal muscle function in heart failure. Proc. Natl. Acad. Sci. USA.102, 9607-12 (2005).

Wehrens et al., Intracellular Calcium Release Channels and Cardiac Disease. Annu. Rev. Physiol. (2004).

Wehrens et al., Ca2+/calmodulin-dependent protein kinase II phosphorylation regulates the cardiac ryanodine receptor. Circ. Res. 94, e61-70 (2004).

Xiang et al., Phosphodiesterase 4D in required for beta2 adrenoceptor subtype-specific signaling in cardiac cyocytes. Proc. Natl. Acad. Sci. USA 102, 909-914 (2005).

Yang et al., A-kinase anchoring protein 100 (AKAP 100) is localized in multiple subcellular compartments in the adult rat heart. J. Cell. Biol. 142, 511-22 (1998).

Yano et al., Altered stoichiometry of FKBP12.6 versus ryanodine receptor as a cause of abnormal Ca²⁺ leak through ryanodine receptor in heart failure. Circulation 102, 2131-36, (2000).

Yano et al., FKBP12.6-Mediated Stablization of Calcium-Release Channel (Ryanodine Receptor) as a Novel Therapeutic Strategy Against Heart Failure. Circulation 107, 477-484, (2003).

Zaccolo et al., Discrete micro domains with high concentration of cAMP in stimulated rat neonatal cardiac myocytes. Science 295, 1711-5 (2002).

What is claimed is:

1. A composition useful for treating or preventing a ryanodine receptor associated disorder comprising:
(a) a phosphodiesterase (PDE)-associated agent; and optionally
(b) a pharmaceutically acceptable carrier.

2. The composition of claim 1, wherein the PDE-associated agent is selected from the group consisting of a PDE protein, a nucleic acid encoding a PDE protein, a member of a PDE signal-transduction pathway, and a modulator of a member of a PDE signal transduction pathway.

3. The composition of claim 2, wherein the PDE-associated agent is PDE4D protein or a nucleic acid encoding PDE4D.

4. The composition of claim 1, wherein the ryanodine receptor associated disorder is a RyR1 associated disorder.

5. The composition of claim 1, wherein the ryanodine receptor associated disorder is a RyR2 associated disorder.

6. The composition of claim 1, wherein the ryanodine receptor associated disorder is a RyR3 associated disorder.

7. A method for treating or preventing a ryanodine receptor associated disorder in a subject comprising augmenting PDE in a ryanodine receptor complex of the subject.

8. The method of claim 7, wherein the PDE is augmented in the ryanodine receptor complex by contacting the ryanodine receptor complex with a PDE-associated agent.

9. The method of claim 8, wherein the PDE-associated agent is selected from the group consisting of a PDE protein, a nucleic acid encoding a PDE, a member of a PDE signal-transduction pathway, and a modulator of a member of a PDE signal transduction pathway.

10. The method of claim 7, wherein the PDE is PDE4D.

11. The method of claim 7, wherein the PDE is PDE4D3.

12. The method of claim 7, wherein the ryanodine receptor is RyR1.

13. The method of claim 7, wherein the ryanodine receptor is RyR2.

14. The method of claim 7, wherein the ryanodine receptor is RyR3.

15. The method of claim 7, wherein the subject is human.

16. The method of claim 7, wherein the ryanodine receptor associated disorder is selected from the group consisting of: cardiac disorders and diseases, skeletal muscular disorders and diseases, cognitive disorders and diseases, malignant hyperthermia, diabetes, and sudden infant death syndrome.

17. The method of claim 16, wherein the cardiac disorder and diseases are selected from the group consisting of irregular heartbeat disorders and diseases; exercise-induced irregular heart beat disorders and diseases; sudden cardiac death; exercise-induced sudden cardiac death; congestive heart failure; chronic obstructive pulmonary disease; and high blood pressure.

18. The method of claim 17, wherein the irregular heartbeat disorders and diseases and exercise-induced irregular heartbeat disorders and diseases are selected from the group consisting of atrial and ventricular arrhythmia; atrial and ventricular fibrillation; atrial and ventricular tachyarrhythmia; atrial and ventricular tachycardia; catechlaminergic polymorphic ventricular tachycardia (CPTV); and exercise-induced variants thereof.

19. The method of claim 16, wherein the skeletal muscular disorder and diseases are selected from the group consisting of skeletal muscle fatigue, exercise-induced skeletal muscle fatigue, muscular dystrophy, bladder disorders, and incontinence.

20. The method of claim 16, wherein the cognitive disorders and diseases are selected from the group consisting of Alzheimer's Disease, forms of memory loss, and age-dependent memory loss.

21. The method of claim 7, wherein the agent increases the expression of PDE gene in the subject.

22. A kit for use in delivering a PDE-associated agent to a ryanodine receptor complex in a subject, comprising: (a) the PDE-associated agent of claim 8; (b) a catheter; and optionally (c) a pharmaceutically acceptable carrier.

23. A method for regulating PKA phosphorylation of a ryanodine receptor comprising contacting the ryanodine receptor complex with an agent that modulates the level of PDE in the ryanodine receptor complex, wherein contacting the ryanodine receptor complex with an agent that increases the level of PDE in the complex results in a reduction of PKA phosporylation of the ryanodine receptor, and contacting the ryanodine receptor complex with an agent that decreases the level of PDE in the complex results in an increase of PKA phosporylation of the ryanodine receptor.

24. The method of claim 23, wherein the agent is selected from the group consisting of a PDE protein, a nucleic acid encoding a PDE, a member of a PDE signal-transduction pathway, and a modulator of a member of a PDE signal transduction pathway.

25. The method of claim 23, wherein the PDE is PDE4D.

26. The method of claim 25, wherein the PDE is PDE4D3.

27. The method of claim 23, wherein the receptor is a RyR1 receptor.

28. The method of claim 23, wherein the receptor is a RyR2 receptor.

29. The method of claim 23, wherein the receptor is a RyR2 receptor.

30. A method for decreasing PKA phosphorylation of a ryanodine receptor comprising contacting the ryanodine receptor complex with an agent that increases the level of PDE in the ryanodine receptor complex.

31. The method of claim 30, wherein the receptor is hyperphosphorylated prior to contacting the ryanodine receptor complex with the agent.

32. The method of claim 30, wherein the agent is selected from the group consisting of a PDE protein, a nucleic acid encoding a PDE, a member of a PDE signal-transduction pathway, and a modulator of a member of a PDE signal transduction pathway.

33. The method of claim 30, wherein the PDE is PDE4D.

34. The method of claim 30, wherein the PDE is PDE4D3.

35. The method of claim 30, wherein the receptor is a RyR1 receptor.

36. The method of claim 30, wherein the receptor is a RyR2 receptor.

37. The method of claim 30, wherein the receptor is a RyR3 receptor

38. A method for regulating Ca²⁺ release and reuptake in the sarcoplasmic reticulum of a cell comprising contacting a ryanodine receptor complex of the cell with an agent that modulates the level of PDE, wherein contacting the ryanodine receptor complex with an agent that increases the level of PDE results in a reduction of Ca²⁺ release from and reuptake into the sarcoplasmic reticulum and contacting the ryanodine receptor complex with an agent that decreases the level of PDE in the complex results in an increase of Ca²⁺ release from and reuptake into the sarcoplasmic reticulum.

39. The method of claim 38, wherein the agent is selected from the group consisting of a PDE protein, a nucleic acid encoding a PDE, a member of a PDE signal-transduction pathway, and a modulator of a member of a PDE signal transduction pathway.

40. The method of claim 38, wherein the PDE is PDE4D.

41. The method of claim 38, wherein the PDE is PDE4D3.

42. The method of claim 38, wherein the receptor is a RyR1 receptor.

43. The method of claim 38, wherein the receptor is a RyR2 receptor.

44. The method of claim 38, wherein the receptor is a RyR3 receptor.

45. A method for decreasing Ca²⁺ release and reuptake in the sarcoplasmic reticulum of a cell comprising contacting a ryanodine receptor complex of the cell with an agent that increases the level of PDE.

46. The method of claim 45, wherein the receptor is hyperphosphorylated prior to contacting the ryanodine receptor complex with the agent.

47. The method of claim 45, wherein the agent is selected from the group consisting of a PDE protein, a nucleic acid encoding a PDE, a member of a PDE signal-transduction pathway, and a modulator of a member of a PDE signal transduction pathway.

48. The method of claim 45, wherein the PDE is PDE4D.

49. The method of claim 45, wherein the PDE is PDE4D3.

50. The method of claim 45, wherein the receptor is a RyR2 receptor.

51. The method of claim 45, wherein the receptor is a RyR1 receptor.
